(19) United States
(12) Patent Application Publication
(10) Pub. No.: US 2022/0380414 A1
(43) Pub. Date: Dec. 1, 2022

(54) COMPOSITIONS AND METHODS BASED ON HIV GP120 MUTANTS

(71) Applicants: CENTRE HOSPITALIER DE L'UNIVERSITÉ DE MONTRÉAL, Montreal (CA); THE TRUSTEES OF THE UNIVERSITY OF PENNSYLVANIA, Philadelphia, PA (US); THE HENRY M. JACKSON FOUNDATION FOR THE ADVANCEMENT OF MILITARY MEDICINE, INC., Bethesda, MD (US)

(72) Inventors: Andrés FINZI, St-Bruno-de-Montarville (CA); Jérémie PRÉVOST, Mirabel (CA); Marzena PAZGIER, Derwood, MD (US); Amos B. SMITH, III, Merion Station, PA (US)

(21) Appl. No.: 17/762,333

(22) PCT Filed: Sep. 23, 2020
(86) PCT No.: PCT/CA2020/051274

§ 371 Date: Mar. 21, 2022

Related U.S. Application Data
(60) Provisional application No. 62/904,821, filed on Sep. 24, 2019.

(51) Int. Cl. C07K 14/16; C07D 285/14; C07D 333/38; G01N 33/569

(52) U.S. Cl. CPC C07K 14/162; C07D 285/14; C07D 333/38; G01N 33/56988

(57) ABSTRACT

Compositions and methods based on the use of mutated HIV-1 gp120 polypeptides having amino acid substitutions at positions 61, 105, 108, 375, 474, 475 and 476 are described. These mutated HIV-1 gp120 polypeptides, which make the HIV env protein more amenable to adopt specific conformations when contacted with gp120 ligands, may be useful as vaccines or tools to identify and characterize agents modulating HIV infection.

### CROSS REFERENCE TO RELATED APPLICATIONS

The present application claims the benefit of U.S. provisional patent application No. 62/904,821 filed Sep. 24, 2019, which is incorporated herein by reference.

### STATEMENT REGARDING FEDERALLY SPONSORED RESEARCH OR DEVELOPMENT

This invention was made with Government support under NIH Grant Nos. R01A1129769 and Contract No. P016M56550/A1150741. The Government has certain rights in this invention.

### TECHNICAL FIELD

The present invention generally relates to Human Immunodeficiency Virus (HIV) infection, and more specifically to compositions and methods for research and therapeutic applications relating to HIV infection.

### BACKGROUND ART

The human immunodeficiency virus type 1 (HIV-1) continues to infect more than 2.1 million individuals annually for an estimated total of 37 million people living with this virus in 2016. Enormous efforts have been made to improve the clinical management of HIV/AIDS through highly-active antiretroviral drugs (HAART). Accordingly, HIV infection can be controlled with HAART which, in most cases, allows for a significant increase in the life expectancy of infected individuals. Unfortunately, HAART is unable to fully restore health or a normal immune status. HAART-treated individuals still experience several co-morbidities including increased cardiovascular disease, bone disorders and cognitive impairment. Most importantly, due to the presence of latent viral reservoirs, persisting mainly in long-lived memory CD4⁺ T cells, therapy interruption leads to the re-emergence of viral replication and AIDS progression. Therefore, the development of new approaches aimed at eradicating or functionally curing HIV infection are desperately needed.

HIV-1 entry is mediated by the interaction of HIV-1 envelope glycoproteins (Env) with the CD4 receptor and either CCR5 or CXCR4 chemokine coreceptors on T cells. Env is exposed on the surface of viral particles and infected cells as three gp120 exterior glycoproteins non-covalently associated with three gp41 transmembrane glycoproteins (gp120-gp41)3(1-3). Binding of gp120 to the CD4 receptor leads to major conformational changes in gp120, resulting in the rearrangement of the V1, V2 and V3 loops, and the formation of the coreceptor binding site (CoRBS) and the bridging sheet (4-11). CD4 interaction also leads to the exposure of a gp41 helical heptad repeat (HR1) (12). Subsequent interaction of gp120 with the coreceptor triggers additional conformational changes in gp41, resulting in the formation of a six-helix bundle formed by HR1 and HR2 heptad repeats and the fusion of viral and cellular membranes (12-14).

Recent studies have shown that, on the surface of intact virions, mature pre-fusion Env transitions from a pre-triggered “closed” conformation (state 1) through a default intermediate conformation (state 2) to an “open” conformation in which it is bound to three CD4 receptor molecules (state 3) (Munro, J. B. et al., Science 346, 759-763 (2014); Herschhorn, A. et al., mBio 7, e01598-e16 (2016); Ma, X. et al., eLife 7, e34271 (2018)). The pre-triggered state 1 conformation of viral Env is preferentially stabilized by many broadly neutralizing antibodies, and thus of interest for the design of immunogens, whereas the state 3 conformation is of interest for the development of small CD4 mimetics or additional type of small inhibitors with the capacity to stabilize Env in more “open” conformations such as states 2, 2A and 3. However, there is currently no approach to induce the viral Env to adopt a given conformation, which would be useful for studying the Env structure as well as for the development of HIV vaccines and entry inhibitors.

The present description refers to a number of documents, the content of which is herein incorporated by reference in their entirety.

### SUMMARY OF THE INVENTION

The present disclosure provides the following items 1 to 32:

1. A composition comprising:

(i) a mutated HIV-1 gp120 polypeptide from an HIV-1 strain, wherein the native residues at positions 61, 105, 108, 375, 474, 475 and 476 in the HIV-1 strain are substituted, and wherein
(a) the HIV-1 strain is a CRF01_AE strain and the native residues at positions 61, 105, 108, 375, 474, 475 and 476 are H, Q, V, H, N, I and K, respectively;
(b) the HIV-1 strain is a Clade A, B, C, D, G or H strain, and the native residues at positions 61, 105, 108, 375, 474, 475 and 476 are Y, H, I, S, D, M and R, respectively;
(c) the HIV-1 strain is a Clade F strain, and the native residues at positions 61, 105, 108, 375, 474, 475 and 476 are Y, H, I, S, N, M and K, respectively;
(d) the HIV-1 strain is a Clade J strain, and the native residues at positions 61, 105, 108, 375, 474, 475 and 476 are Y, H, I, S, D, M and K, respectively; or
(e) the HIV-1 strain is a Clade K strain, and the native residues at positions 61, 105, 108, 375, 474, 475 and 476 are Y, H, I, I, D, M and R, respectively;
   and

(ii) a gp120 or gp41 ligand.

2. The composition of item 1, wherein the HIV-1 strain is a CRF01_AE strain and the mutated HIV-1 gp120 polypeptide comprises one or more of the following substitutions: H61Y, Q105H, V1081, H375T or H375S, N474D, 1475M, and K476R.
3. The composition of item 2, wherein the mutated HIV-1 gp120 polypeptide comprises the following substitutions: (1) H61Y, (2) Q105H, (3) V1081, (4) H375T, (5) N474D, (6) 1475M, and (7) K476R.
4. The composition of item 2, wherein the mutated HIV-1 gp120 polypeptide comprises the following substitutions: (1) H61Y, (2) Q105H, (3) V1081, (4) H375S, (5) N474D, (6) 1475M, and (7) K476R.
5. The composition of item 1, wherein the HIV-1 strain is a clade A, B, C, D, G or H HIV-1 strain, and the mutated HIV-1 gp120 polypeptide comprises the following substitutions: Y61H, H105Q, 1108V, S375H, D474N, M4751, and R476K.
6. The composition of item 1, wherein the HIV-1 strain is a clade F HIV-1 strain, and the mutated HIV-1 gp120 polypeptide comprises the following substitutions: Y61H, H105Q, 1108V, S375H, N474D, M4751, and K476R.
7. The composition of item 1, wherein the HIV-1 strain is a clade J HIV-1 strain, and the mutated HIV-1 gp120 polypeptide comprises the following substitutions: Y61H, H105Q, 1108V, S375H, D474N, M4751, and K476R.
8. The composition of item 1, wherein the HIV-1 strain is a clade K HIV-1 strain, and the mutated HIV-1 gp120 polypeptide comprises the following substitutions: Y61H, H105Q, 1108V, 1375H, D474N, M4751, and R476K.
9. The composition of any one of items 1 to 8, wherein the mutated HIV-1 gp120 polypeptide is an HIV envelope trimer.
10. The composition of any one of items 1 to 9, wherein the gp120 ligand induces said Env trimer into an open state 2/3 conformation.
11. The composition of item 10, wherein the gp120 ligand is a CD4 mimetic (CD4mc).
12. The composition of item 11, wherein said CD4mc is the following compound:

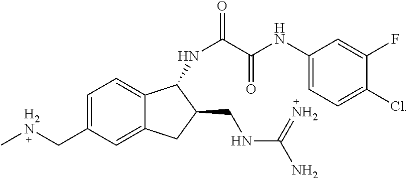

13. The composition of any one of items 1 to 9, wherein the gp120 ligand induces said Env trimer into a closed state 1 conformation.
14. The composition of item 13, wherein the gp120 ligand is a conformational blocker.
15. The composition of item 13 or 14, wherein the gp120 ligand is one of the following compounds:

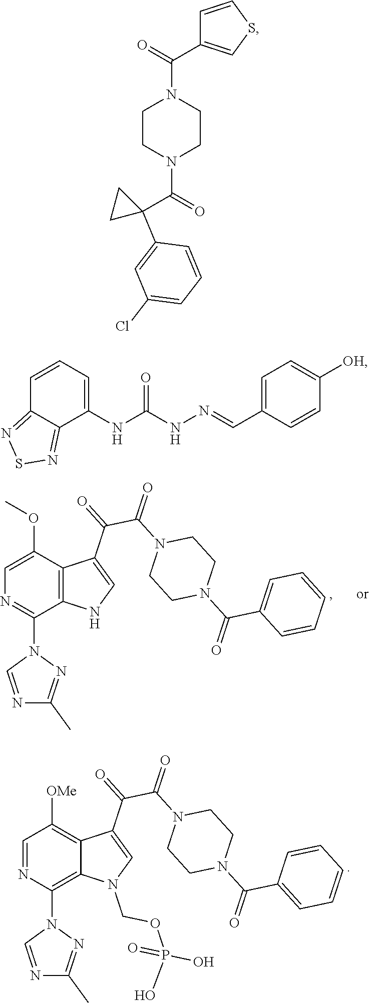

16. The composition of any one of items 1 to 15, further comprising a vaccine adjuvant.
17. The composition of any one of items 1 to 16, wherein said mutated HIV-1 gp120 polypeptide is comprised in a cell, a liposome or a virus-like particle (VLP).
18. A method for eliciting an immune response to HIV-1 in a subject, comprising administering to the subject a prophylactically or therapeutically effective amount of (i) the mutated HIV-1 gp120 polypeptide defined in any one of items 1 to 9 and 17, and (ii) a gp120 ligand.
19. The method of item 18, comprising administering to the subject a prophylactically or therapeutically effective amount of the composition of any one of items 1 to 17.
20. The method of item 18 or 19, wherein said subject is not infected by HIV-1.
21. The method of item 18 or 19, wherein said subject is infected by HIV-1.
22. A method for determining whether a test agent binds to an HIV Env trimer into an open state 2/3 conformation comprising contacting said test agent with the mutated HIV-1 gp120 polypeptide defined in any one of items 1 to 9 and 17, and the gp120 ligand defined in any one of items 10 to 12.
23. A method for determining whether a test agent binds to an HIV Env trimer into a closed state 1 conformation comprising contacting said test agent with the mutated HIV-1 gp120 polypeptide defined in any one of items 1 to 9 and 17, and the gp120 ligand defined in any one of items 13 to 15.
24. A method for inducing an HIV Env trimer into an open state 2/3 conformation comprising contacting an HIV Env trimer comprising the mutated HIV-1 gp120 polypeptide defined in any one of items 1 to 9 and 17 with the gp120 ligand defined in any one of items 10 to 12.
25. A method for inducing an HIV Env trimer into an open state 1 conformation comprising contacting an HIV Env trimer comprising the mutated HIV-1 gp120 polypeptide defined in any one of items 1 to 9 and 17 with the gp120 ligand defined in any one of items 13 to 15.
26. The method of item 22 or 24, comprising the mutated HIV-1 gp120 polypeptide defined in item 3.
27. The method of item 23 or 25, comprising the mutated HIV-1 gp120 polypeptide defined in item 4.
28. A method for determining whether a test agent induces a closed (state 1) conformation of an HIV Env trimer comprising (a) contacting the mutated HIV-1 gp120 polypeptide defined in any one of items 1 to 9 and 17 with said test agent, and (b) determining whether the HIV Env trimer is in a closed (state 1) conformation.
29. A method for determining whether a test agent induces an open (state 2/3) conformation of an HIV Env trimer comprising (a) contacting the mutated HIV-1 gp120 polypeptide defined in any one of items 1 to 9 and 17 with said test agent, and (b) determining whether the HIV Env trimer is in an open (state 2/3) conformation.
30. Use of (i) the mutated HIV-1 gp120 polypeptide defined in any one of items 1 to 9 and 13, and (ii) a gp120 ligand, for eliciting an immune response to HIV-1 in a subject.
31. Use of (i) the mutated HIV-1 gp120 polypeptide defined in any one of items 1 to 9 and 13, and (ii) a gp120 ligand, for the manufacture of a medicament for eliciting an immune response to HIV-1 in a subject.
32. A complex or composition comprising (i) the mutated HIV-1 gp120 polypeptide defined in any one of items 1 to 9 and 13, and (ii) a gp120 ligand, for use in eliciting an immune response to HIV-1 in a subject.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1A shows a sequence alignment of selected gp120 residues located in the Phe43 cavity (375) or the inner domain layers (61, 105, 108, 474, 475, 476) based on Env consensus sequence of CRF01_AE strains and each HIV-1 group M clades. The 2017 Los Alamos database-curated Env alignment was used as the basis for this figure, which contains 5,471 amino acid HIV-1 group M sequences (including 481 of CRF01_AE, 220 of subtype A1, 1,937 of subtype B and 1,377 of subtype C). Residue numbering is based on that of the HXBc2 strain of HIV-1 (Korber B. et al., 1998. Human Retroviruses and AIDS pp. III:102-111). Identical residues are shaded in dark gray, and non-identical residues are highlighted in light gray. HQVHNIK (SEQ ID NO:1); YHISDMR (SEQ ID NO:2); YHISNMK (SEQ ID NO:3); YHISDMK (SEQ ID NO:4); YHIIDMR (SEQ ID NO:5).

FIGS. 1B-F show logo depictions of the frequency of each amino acid from the Phe43 cavity at positions 366 to 378 in isolates from all HIV-1 clades and CRFs (B), CRF01_AE (C), clade A1 (D), clade B (E), clade C (F). The height of the letter indicates its frequency within the clade. The box beside each logo indicates the frequency of all the amino acids at position 375. Logo plots (Crooks G E, et al. 2004. Genome Res 14:1188-1190) for HIV were made using the Analyze Align tool at the HIV database and are based on the WebLogo 3 program (https://www.hiv.lanl.gov/content/sequence/ANALYZEALIGN/analyze align.html) and the HIV-1 database global curated and filtered 2017 alignment published circa June 2018.

FIGS. 2A-G show the effect of gp120 layer mutations (LM) on neutralization by soluble CD4 and CD4-mimetic compounds. Recombinant HIV-1 strains expressing luciferase and bearing wild-type or mutant CRF01_AE Envs (92TH023 and CM244 isolates (Montefiori D C et al. 2012. J Infect Dis 206:431-441; Zoubchenok D, et al. 2017. J Virol 91) were normalized by reverse transcriptase activity. Normalized amounts of viruses were incubated with serial dilutions of soluble CD4 (sCD4) (Finzi A, et al. 2010. Mol Cell 37:656-667) (FIG. 2A, B), small CD4 mimetic (CD4mc) BNM-III-170 (Melillo B, et al. 2016. ACS Med Chem Lett 7:330-334) (FIG. 2C, D), or CD4-mimetic peptide M48U1 (Martin L et al. 2003. Nat Biotechnol 21:71-76) (FIG. 2E, F) at 37° C. for 1 h prior to infection of Cf2Th-CD4/CCR5 cells (LaBonte J A et al. 2000. J Virol 74:10690-10698). Infectivity at each dilution of sCD4 or CD4mc tested is shown as the percentage of infection without sCD4 or CD4mc for each particular mutant. Quadruplicate samples were analyzed in each experiment. Data shown are the means of results obtained in at least 3 independent experiments. The error bars represent the standard deviations. Neutralization half maximal inhibitory concentration (IC₅₀) are summarized in FIG. 2G.

FIGS. 3A-G show the effect of single gp120 layer mutations on neutralization by sCD4 or CD4mc. Recombinant HIV-1 strains expressing luciferase and bearing wild-type or mutant CRF01_AE Envs (92TH023 and CM244 isolates) were normalized by reverse transcriptase activity. Normalized amounts of viruses were incubated with serial dilutions of sCD4 (FIG. 3A, B), BNM-III-170 (FIG. 3C, D), or M48U1 (FIG. 3E, F) at 37° C. for 1 h prior to infection of Cf2Th-CD4/CCR5 cells. Infectivity at each dilution of sCD4 or CD4mc tested is shown as the percentage of infection without sCD4 or CD4mc for each particular mutant. Quadruplicate samples were analyzed in each experiment. Data shown are the means of results obtained in at least 3 independent experiments. The error bars represent the standard deviations. Neutralization half maximal inhibitory concentration (IC₅₀) are summarized in FIG. 3G.

FIGS. 4A-F show that Phe43 cavity and inner domain (LM) changes render CRF01_AE strain susceptible to CD4mc-induced Env conformational changes. Cell-surface staining of HEK293T cells transfected with different CRF01_AE Env expressors (92TH023 and CM244 isolates) WT or their mutated counterparts together with a GFP expressor (in order to identify positively transfected cells) using a panel of Env ligands. (FIG. 4A, B) sCD4 binding in presence of BNM-III-170 (50 μM) or not (DMSO) was detected with the anti-CD4 OKT4 mAb. Binding of broadly-neutralizing antibodies (bNAbs) that preferentially recognize state 1 Env (Lu M et al. 2019. Nature 568, pages 415-419; Munro J B et al., 2014. Science 346:759-763) (FIG. 4B, E) and non-neutralizing antibodies (nnAbs) that preferentially recognize states 2/2A/3 (Alsahafi N et al., 2019. Cell Host Microbe 25:578-587, Prevost J et al., 2018. Virology 515:38-45) (FIG. 4C, F) was also performed in presence of BNM-III-170 (50 μM) or not (DMSO). Shown are the mean fluorescence intensities (MFI) obtained in presence of BNM-III-170 normalized to the MFI in absence of BNM-111-170 (DMSO) from the transfected (GFP+) population for staining obtained in at least 3 independent experiments. All MFI were normalized to MFI signals obtained with the anti-gp120 outer domain 2G12. Decrease in sCD4 signal indicates competition with BNM-III-170 (i.e., this represents an indirect measure of BNM-III-170 interaction with each Env mutant) (FIGS. 4A and D). Decrease in bNAbs binding suggest an “opening” of Env induced by BNM-III-170 (FIGS. 4B and E). Increase in nnAbs signal is consistent with the capacity of BNM-III-170 to push Env to downstream “open” states 2/3 conformations (C and F). Note that LM-HS and LM-HT are, among all tested mutants, the more susceptible to BNM-III-170 induced conformational changes. Error bars indicate mean±SEM. Statistical significance was tested using an unpaired t-test (* p<0.05, ** p<0.01, *** p<0.001, **** p<0.0001, ns: non significant).

FIGS. 5A-B show that Phe43 cavity and inner domain changes enhance the sensitivity of CRF01_AE strains to neutralization by a cyclic peptide triazole. Recombinant HIV-1 strains expressing luciferase and bearing wild-type or mutant CRF01_AE Envs (92TH023 isolate) were normalized by reverse transcriptase activity. (FIG. 5A) Normalized amounts of viruses were incubated with serial dilutions of the cyclic peptide triazoles (cPT) AAR029N2 (Rashad A A et al., 2017. Org Biomol Chem 15:7770-7782) at 37° C. for 1 h prior to infection of Cf2Th-CD4/CCR5 cells. Infectivity at each dilution of cPT tested is shown as the percentage of infection without cPT for each particular mutant. Quadruplicate samples were analyzed in each experiment. Data shown are the means of results obtained in at least 3 independent experiments. The error bars represent the standard deviations. Neutralization half maximal inhibitory concentration (IC₅₀) are summarized in (FIG. 5B).

FIGS. 6A-F show the effect of Phe43 cavity and layer mutations (LM) on neutralization by small molecules known to stabilize Env State 1. Recombinant HIV-1 strains expressing luciferase and bearing wild-type or mutant CRF01_AE Envs (92TH023 and CM244 isolates) were normalized by reverse transcriptase activity. Normalized amounts of viruses were incubated with serial dilutions of BMS-626529 (Nowicka-Sans B et al. 2012. Antimicrob Agents Chemother 56:3498-3507; Pancera M et al., 2017. Nat Chem Biol 13:1115-1122) (FIG. 6A, B) or 484 (16) (FIG. 6C) at 37° C. for 1 h prior to infection of Cf2Th-CD4/CCR5 cells. Infectivity at each dilution of the compound tested is shown as the percentage of infection without the compound for each particular mutant. Quadruplicate samples were analyzed in each experiment. Data shown are the means of results obtained in at least 3 independent experiments. The error bars represent the standard deviations. Neutralization half maximal inhibitory concentration (IC₅₀) are summarized in FIG. 6D. (FIG. 6E, F) Cell-surface staining of 293T cells transfected with different CRF01_AE Env expressors (92TH023 and CM244 isolates) WT or their mutated counterparts together with a GFP expressor (in order to identify positively transfected cells) using sCD4. Binding of sCD4 in presence of BMS-626529 (50 μM) or not (DMSO) was detected with the anti-CD4 OKT4 mAb. Shown are the mean fluorescence intensities (MFI) obtained in presence of BNM-III-170 normalized to the MFI in absence of BNM-III-170 (DMSO) from the transfected (GFP+) population for staining obtained in at least 3 independent experiments. All MFI were normalized to 2G12 MFI for each Env mutants. Decrease in sCD4 signal indicates competition with BMS-626529 (i.e., this represents an indirect measure of BMS-626529 interaction with each Env mutant). Note the statistically significant decrease for the LM-HS mutant. Error bars indicate mean±SEM. Statistical significance was tested using an unpaired t-test (** p<0.01, ns: non significant).

FIGS. 7A-H show that Phe43 cavity and inner domain layer (LM) changes enhance susceptibility of CRF01_AE strain to small molecules stabilizing States 1, 2/3. Cell-surface staining of primary CD4⁺ T cells infected with SHIV expressing a CRF01_AE Env from a transmitted founder strain (40100 isolate) (Chenine A L et al., 2018. J Acquir Immune Defic Syndr 78:348-355; Kijak G H et al., 2017. PLoS Pathog 13:e1006510) mutated to harbor the LM+HS mutations using a panel of Env ligands. Binding of anti-Env mAbs preferring State 1 (PGT128, PGT145, PG9, 10-1074) (FIG. 7A-D) or preferring State 2/3 (19b, 17b) (FIG. 7E-F) was performed in presence of DMSO or increasing amount of BNM-III-170 (stabilises States 2/3 (Munro J B et al., 2014. Science 346:759-763; Alsahafi N et al., 2019. Cell Host Microbe 25:578-587: e575; Herschhorn A, et al. 2017. Nat Commun 8:1049) or BMS-626529 (stabilizes State 1, (Lu M et al. 2019. Nature 568, pages 415-419; Munro J B et al., 2014. Science 346:759-763)). Shown are the mean fluorescence intensities (MFI) obtained in presence of the compounds normalized to the MFI in absence of any compound (DMSO) from the infected (p27+) population. All MFI were normalized to 2G12 MFI for each mAb. State 1 and State 2/3 Abs were grouped in FIG. 7G and FIG. 7H, respectively.

FIGS. 8A-G show the effect of gp120 layers mutations (LM) on the CD4 binding site. (FIG. 8A, B) Cell-surface staining of 293T cells transfected with CRF01_AE Env expressors (92TH023 and CM244 isolates) WT or their mutated counterparts together with a GFP expressor (in order to identify positively transfected cells) using a panel of CD4-binding site antibodies (CD4BS Abs). Shown are the mean fluorescence intensities (MFI) normalized to 2G12 MFI obtained in the transfected (GFP′) population for staining obtained in at least 3 independent experiments. All MFI were normalized to 2G12 MFI for each Env mutants. Error bars indicate mean±SEM. Statistical significance was tested using a paired t-test or a Wilcoxon signed-rank test based on statistical normality (* p<0.05, ** p<0.01, *** p<0.001). (FIG. 8C-F) Recombinant HIV-1 strains expressing luciferase and bearing wild-type or mutant CRF01_AE Envs (92TH023 and CM244 isolates) were normalized by reverse transcriptase activity. Normalized amounts of viruses were incubated with serial dilutions of four different CD4BS bNAbs: VRC01 (FIG. 8C), VRC16 (FIG. 8D), VRC13 (FIG. 8E), or b12 (FIG. 8F) at 37° C. for 1 h prior to infection of Cf2Th-CD4/CCR5 cells. Infectivity at each dilution of mAbs tested is shown as the percentage of infection without antibody for each particular mutant. Quadruplicate samples were analyzed in each experiment. Data shown are the means of results obtained in at least 3 independent experiments. The error bars represent the standard deviations. Neutralization half maximal inhibitory concentration (IC₅₀) are summarized in FIG. 8G.

FIG. 9 shows the structure of representative HIV-1 entry inhibitors (conformational blockers) disclosed in U.S. Pat. Nos. 7,745,625, 8,168,615, 8,461,333 and 8,871,771 and in PCT publication No. WO 2005/090367.

FIGS. 10A-K show the structure of representative CD4mc compounds disclosed in PCT publication No. WO/2020/028482.

FIG. 11A shows the amino acid sequence of Envelope glycoprotein gp160 from the HXBc2 strain of HIV-1 (UniProtKB accession No. P04578.2, SEQ ID NO:6), with residues 61, 105, 108, 375, 474, 475 and 476 in bold and underlined. Residues 1-32 correspond to the signal peptide, residues 33-511 correspond to the sequence of surface protein gp120 and residues 512-856 correspond to the sequence of transmembrane protein gp41.

FIGS. 11B-D show an alignment of the amino acid sequence of Envelope glycoprotein gp160 from the HXBc2 strain of HIV-1 and that of the consensus gp160 sequence from HIV clade A1 (SEQ ID NO:7), A2 (SEQ ID NO:8), B (SEQ ID NO:9), C (SEQ ID NO:10), D (SEQ ID NO:11), F1 (SEQ ID NO:12), F2 (SEQ ID NO:13), G (SEQ ID NO:14), and H (SEQ ID NO:15), with the residues corresponding to residues 61, 105, 108, 375, 474, 475 and 476 of HXBc2 gp160 in bold and underlined.

FIGS. 12A-B show the structure of representative CD4mc compounds disclosed in PCT publication No. WO 2013/090696.

### DISCLOSURE OF INVENTION

In the studies described herein, the present inventors have shown that introducing certain mutations in the HIV Env protein “re-shapes” the Phe43 cavity and makes the HIV env protein amenable to adopt specific conformations when contacted with gp120 ligands. More specifically, full-length gp160 glycoprotein constructs from CRF01_AE strains comprising the six mutations H61Y, Q105H, V1081, N474D, 1475M, and K476R, combined with H375T, tend to adopt an open state 2/3 configuration in the presence of the CD4 mimetic BNM-III-170, whereas full-length gp160 glycoprotein constructs from CRF01_AE strains comprising the same six mutations, combined with H375S, tend to adopt a state 1 configuration in the presence of the HIV-1 attachment inhibitor Temsavir (BMS-626529), a conformational blocker.

Accordingly, in a first aspect, the present disclosure provides a complex or composition comprising:

(i) a mutated HIV-1 gp120 polypeptide from an HIV-1 strain, wherein the native residues at positions 61, 105, 108, 375, 474, 475 and 476 in the HIV-1 strain (numbering based on the amino acid sequence of Envelope glycoprotein gp160 from the HXBc2 strain of HIV-1, UniProtKB accession No. P04578.2, SEQ ID NO:6) are substituted, and wherein (a) the HIV-1 strain is a CRF01_AE strain and the native residues at positions 61, 105, 108, 375, 474, 475 and 476 are H, Q, V, H, N, I and K, respectively; (b) the HIV-1 strain is a Clade A, B, C, D, G or H strain, and the native residues at positions 61, 105, 108, 375, 474, 475 and 476 are Y, H, I, S, D, M and R, respectively; (c) the HIV-1 strain is a Clade F strain, and the native residues at positions 61, 105, 108, 375, 474, 475 and 476 are Y, H, I, S, N, M and K, respectively; (d) the HIV-1 strain is a Clade J strain, and the native residues at positions 61, 105, 108, 375, 474, 475 and 476 are Y, H, I, S, D, M and K, respectively; or (e) the HIV-1 strain is a Clade K strain, and the native residues at positions 61, 105, 108, 375, 474, 475 and 476 are Y, H, I, I, D, M and R, respectively; and

(ii) a gp120 or gp41 ligand.

In another aspect, the present disclosure provides a method for rendering an HIV Env trimer from an HIV strain more amenable to adopt a closed or open conformation following binding of a gp120 or gp41 ligand, comprising introducing amino acid substitutions at positions 61, 105, 108, 375, 474, 475 and 476 in the gp120 protein forming said HIV Env trimer.

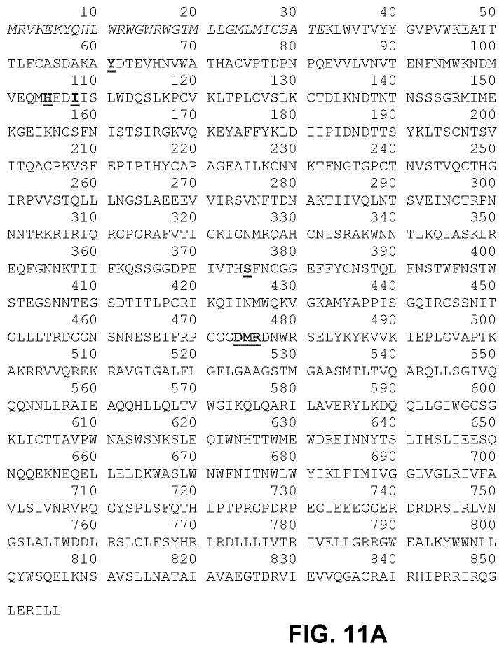

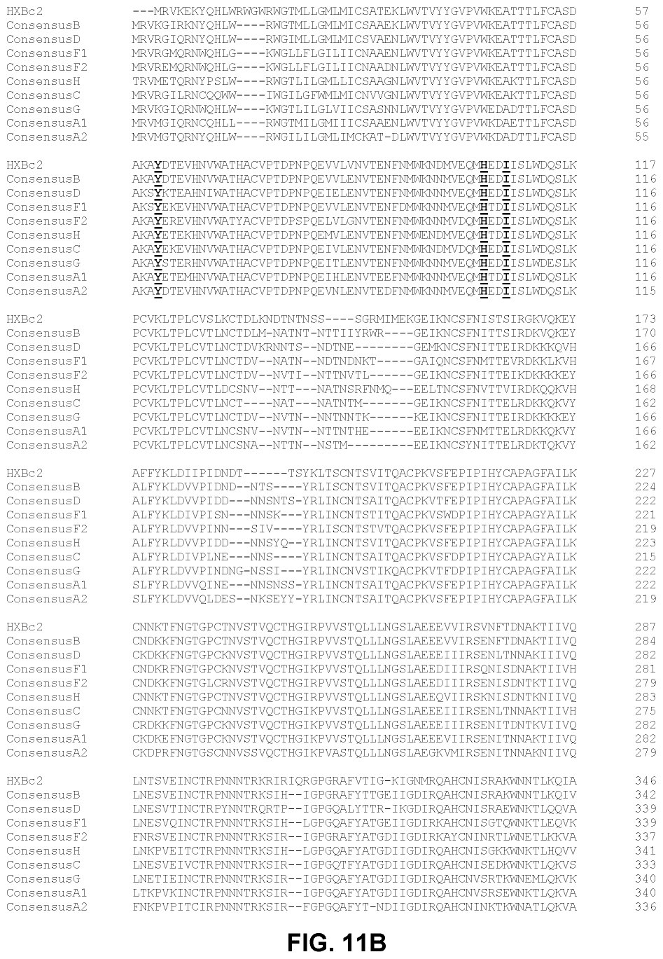

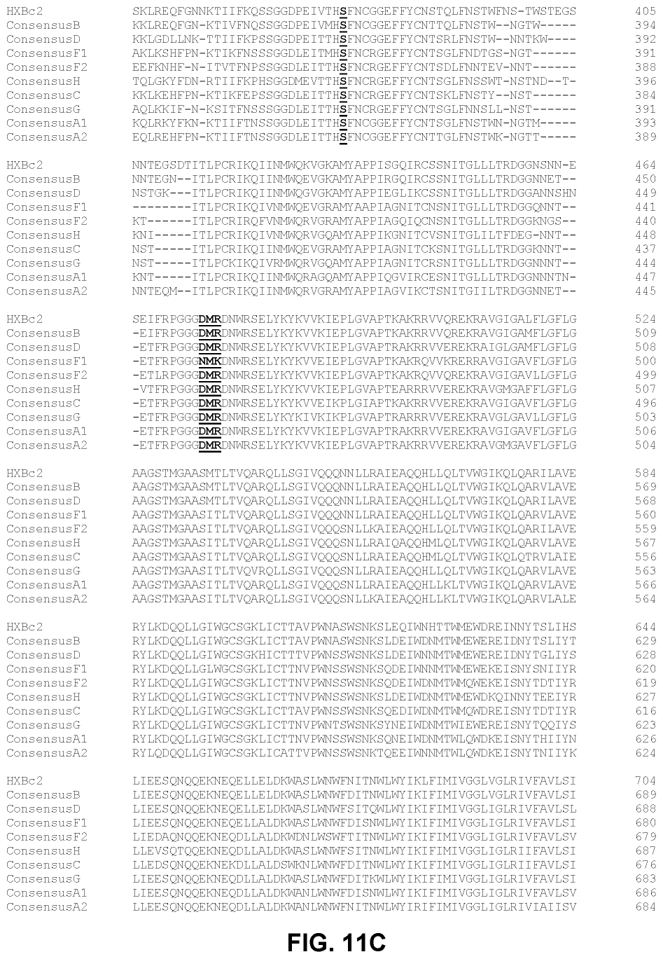

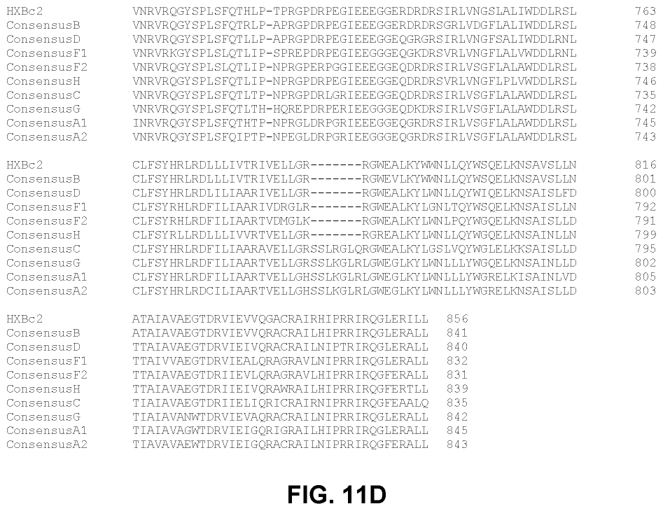

The numbering used in the disclosed HIV-1 Env proteins is relative to the HXBc2 strain of HIV-1 (UniProtKB/Swiss-Prot: P04578.2, FIG. 11A). The corresponding residues in the Env proteins from other HIV strains or clades may be easily determined by aligning their sequences with that of the Env protein from HXBc2 (FIG. 11A). FIGS. 11B-D disclose an alignment of the consensus Env sequences from several HIV clades with that of HXBc2.

As used herein, the term “clade” refers to related human immunodeficiency viruses (HIVs) classified according to their degree of genetic similarity. A clade generally refers to a distinctive branch in a phylogenetic tree. There are currently four major groups of HIV-1 isolates: M, N, O and P. Group M (the Main group) is responsible for the majority of cases in the global pandemic and consists of 9 major clade subtypes (A1, A2, B, C, D, F1, F2, G, H, J, and K) and many circulating recombinant forms (CRFs).

In an embodiment, the substitution is with an amino acid that is present at high frequency (e.g., more than 30%, 40% or 50%, preferably more than 60%) at the corresponding position in another HIV strain or clade.

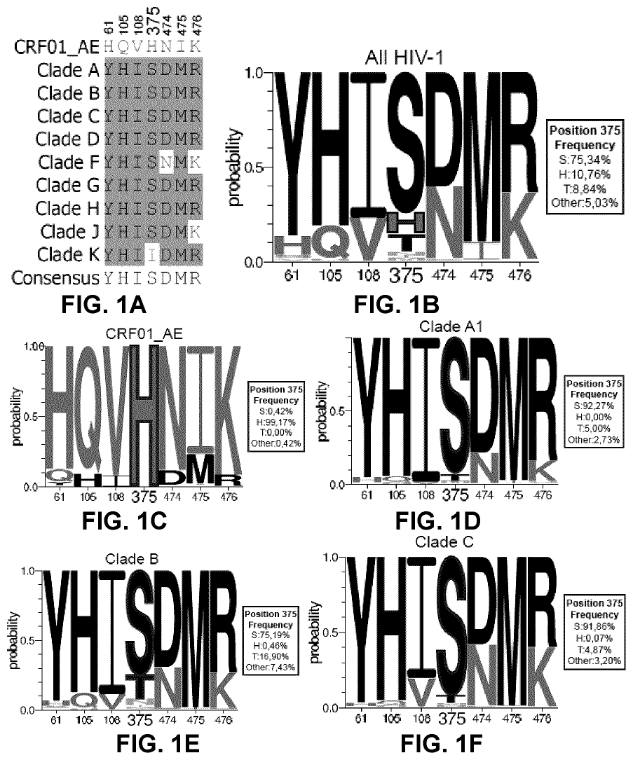

In an embodiment, the mutated HIV-1 gp120 polypeptide is from a circulating recombinant form (CRF), more specifically CRF01_AE. As shown in FIG. 1, the native residues at positions 61, 105, 108, 375, 474, 475 and 476 in gp120 of CRF01_AE strain are H61, Q105, V108, H375, N474, 1475 and K476, but the corresponding residues found at high frequency (e.g., more than 50%) in other clades are Y61, H105, 1108, S375 or T375, D474, M475 and R476. Accordingly, in an embodiment, the HIV-1 strain is a CRF01_AE strain, and the mutated HIV-1 gp120 polypeptide comprises the following substitutions: H61Y, Q105H, V1081, H375T or H375S, N474D, 1475M, and K476R. In an embodiment, the HIV-1 strain is a CRF01_AE strain, and the mutated HIV-1 gp120 polypeptide comprises an amino acid sequence having at least 70, 75, 80, 85, 90, 95, 96, 97, 98 or 99% sequence identity with residues 33-502 of SEQ ID NO: 15.

In another embodiment, the mutated HIV-1 gp120 polypeptide is from a clade A, B, C, D, G or H HIV-1 strain, and the mutated HIV-1 gp120 polypeptide comprises the following substitutions: Y61H, H105Q, 1108V, S375H, D474N, M4751, and R476K.

In another embodiment, the mutated HIV-1 gp120 polypeptide is from a clade F HIV-1 strain, and the mutated HIV-1 gp120 polypeptide comprises the following substitutions: Y61H, H105Q, 1108V, S375H, N474D, M4751, and K476R.

In another embodiment, the mutated HIV-1 gp120 polypeptide is from a clade J HIV-1 strain, and the mutated HIV-1 gp120 polypeptide comprises the following substitutions: Y61H, H105Q, 1108V, S375H, D474N, M4751, and K476R.

In another embodiment, the mutated HIV-1 gp120 polypeptide is from a clade K HIV-1 strain, and the mutated HIV-1 gp120 polypeptide comprises the following substitutions: Y61H, H105Q, 1108V, 1375H, D474N, M4751, and R476K.

In another embodiment, the mutated HIV-1 gp120 polypeptide is from a clade A and comprises an amino acid sequence having at least 70, 75, 80, 85, 90, 95, 96, 97, 98 or 99% sequence identity with residues 33-493 of SEQ ID NO: 7 (clade A1) or residues 32-491 of SEQ ID NO: 8 (clade A2).

In another embodiment, the mutated HIV-1 gp120 polypeptide is from a clade B and comprises an amino acid sequence having at least 70, 75, 80, 85, 90, 95, 96, 97, 98 or 99% sequence identity with residues 33-496 of SEQ ID NO: 9.

In another embodiment, the mutated HIV-1 gp120 polypeptide is from a clade C and comprises an amino acid sequence having at least 70, 75, 80, 85, 90, 95, 96, 97, 98 or 99% sequence identity with residues 33-483 of SEQ ID NO: 10.

In another embodiment, the mutated HIV-1 gp120 polypeptide is from a clade D and comprises an amino acid sequence having at least 70, 75, 80, 85, 90, 95, 96, 97, 98 or 99% sequence identity with residues 33-495 of SEQ ID NO: 11.

In another embodiment, the mutated HIV-1 gp120 polypeptide is from a clade F and comprises an amino acid sequence having at least 70, 75, 80, 85, 90, 95, 96, 97, 98 or 99% sequence identity with residues 33-487 of SEQ ID NO: 12 (clade F1) or 33-486 of SEQ ID NO: 13 (clade F2).

In another embodiment, the mutated HIV-1 gp120 polypeptide is from a clade G and comprises an amino acid sequence having at least 70, 75, 80, 85, 90, 95, 96, 97, 98 or 99% sequence identity with residues 33-490 of SEQ ID NO: 14.

In another embodiment, the mutated HIV-1 gp120 polypeptide is from a clade H and comprises an amino acid sequence having at least 70, 75, 80, 85, 90, 95, 96, 97, 98 or 99% sequence identity with residues 33-494 of SEQ ID NO: 15.

“Identity” refers to sequence identity between two polypeptides. Identity can be determined by comparing each position in the aligned sequences. Methods of determining percent identity are known in the art, and several tools and programs are available to align amino acid sequences and determine a percentage of identity including EMBOSS Needle, ClustalW, SIM, DIALIGN, etc. As used herein, a given percentage of identity with respect to a specified subject sequence, or a specified portion thereof, may be defined as the percentage of amino acids in the candidate derivative sequence identical with the amino acids in the subject sequence, after aligning the sequences and introducing gaps, if necessary to achieve the maximum percent sequence identity, as generated by the Smith Waterman algorithm (Smith & Waterman, J. Mol. Biol. 147: 195-7 (1981)) using the BLOSUM substitution matrices (Henikoff & Henikoff, Proc. Natl. Acad. Sci. USA 89:10915-9 (1992)) as similarity measures. A “% identity value” is determined by the number of matching identical amino acids divided by the sequence length for which the percent identity is being reported.

The term gp120 or gp41 ligand as used herein refers to a molecule (e.g., small molecule, peptide, antibody or antigen-binding fragment thereof, etc., either synthetic or natural) that binds to gp120 and/or gp41. In an embodiment, the gp120 or gp41 ligand is a synthetic molecule.

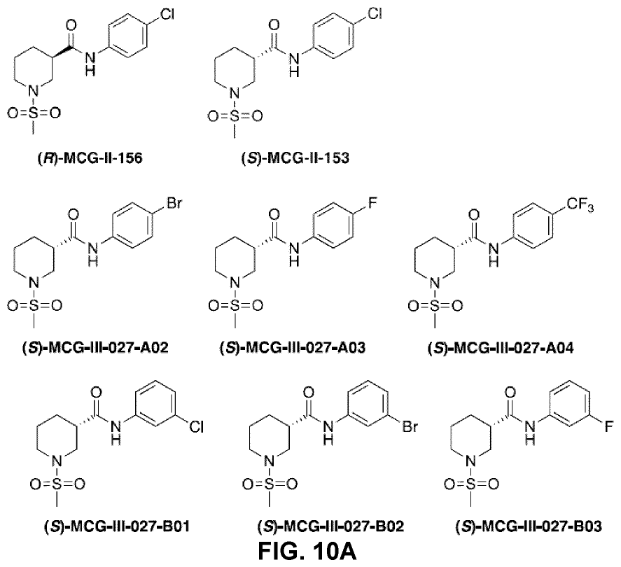

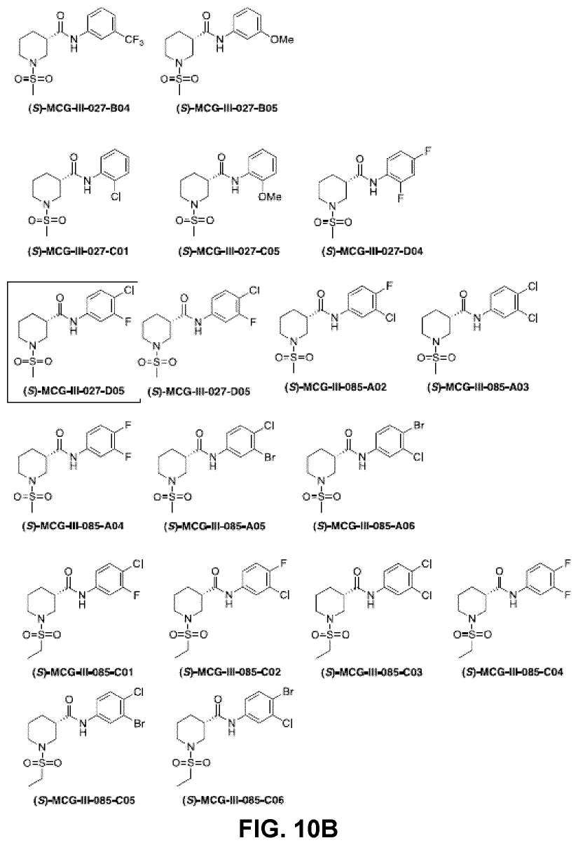

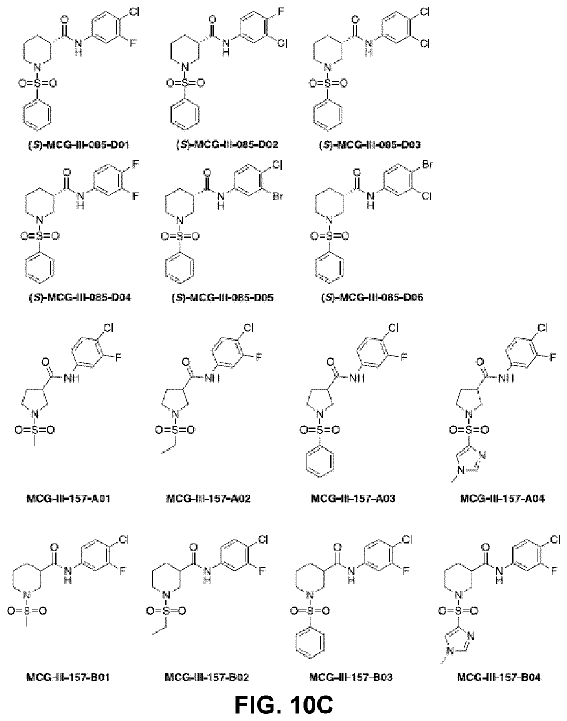

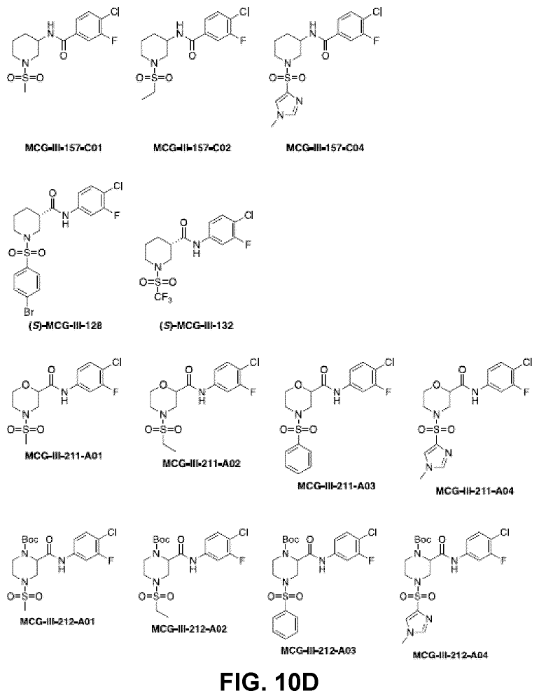

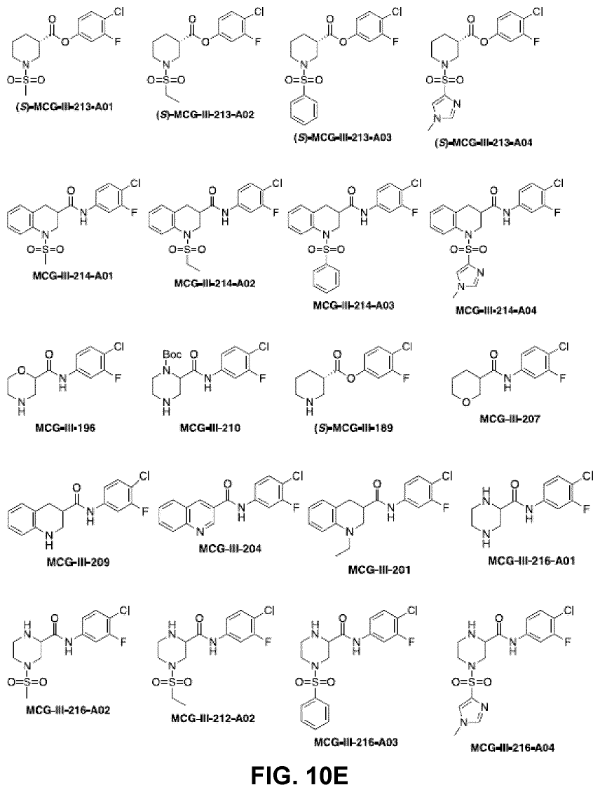

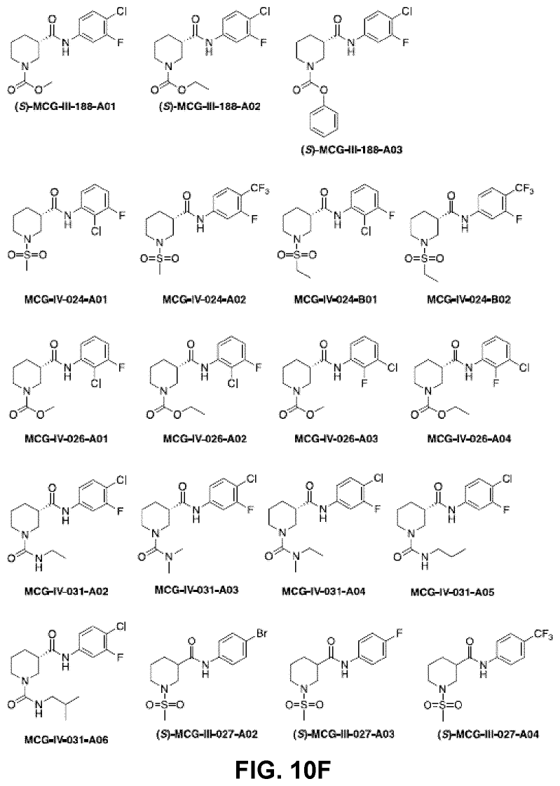

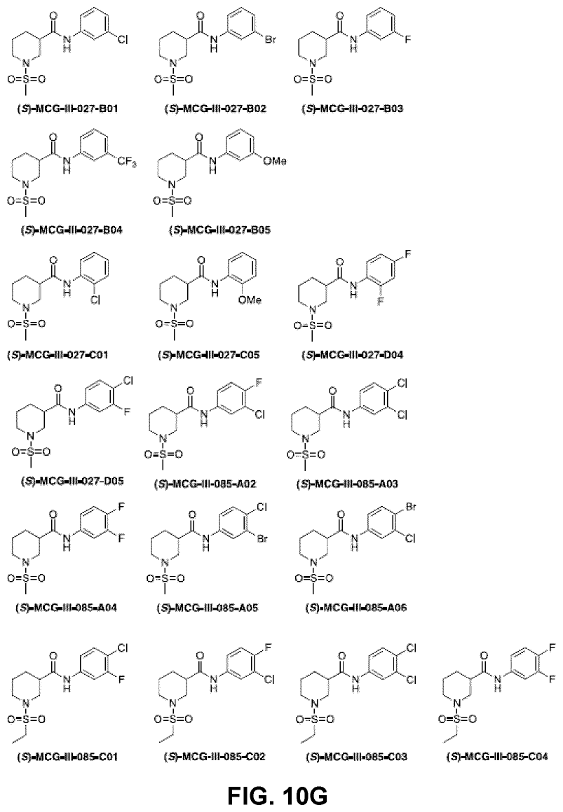

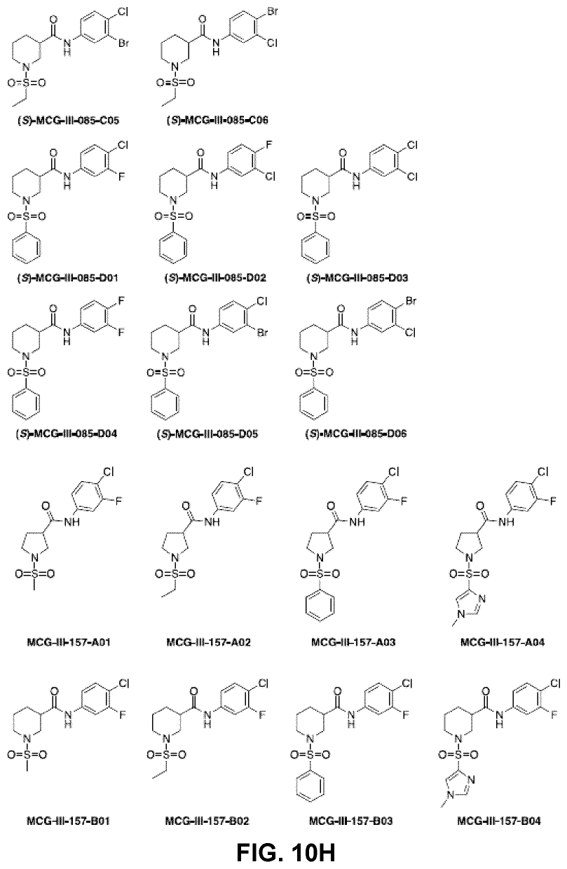

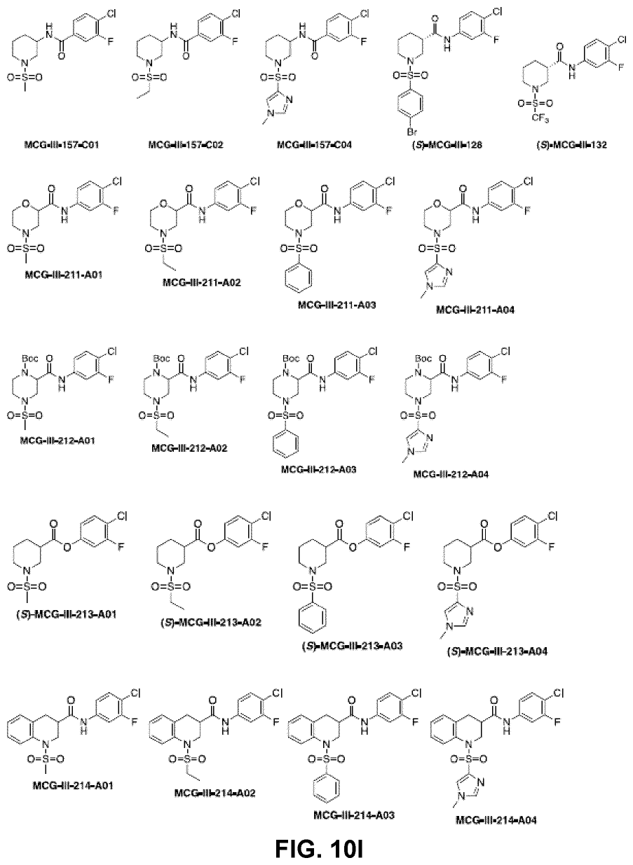

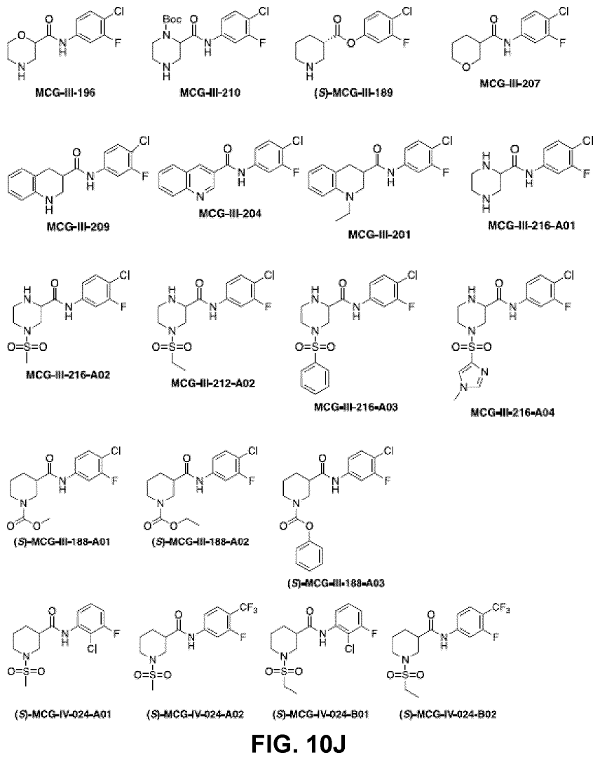

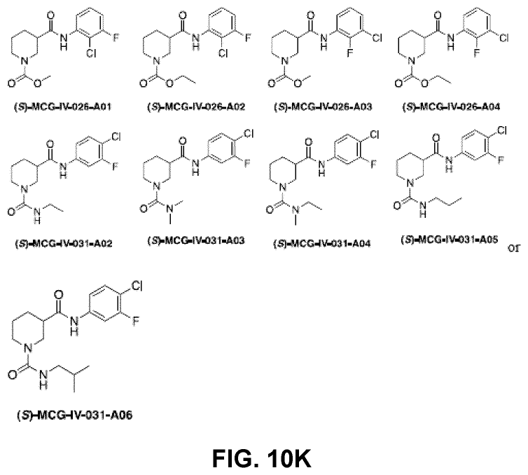

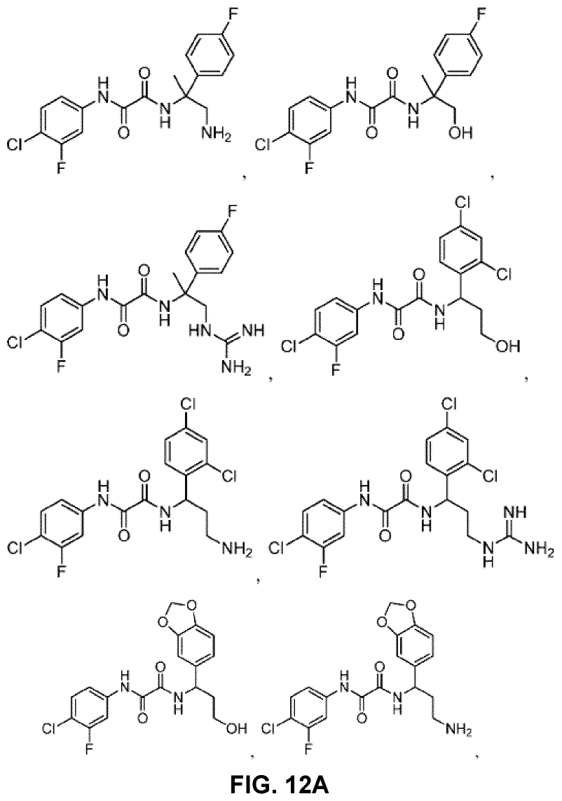

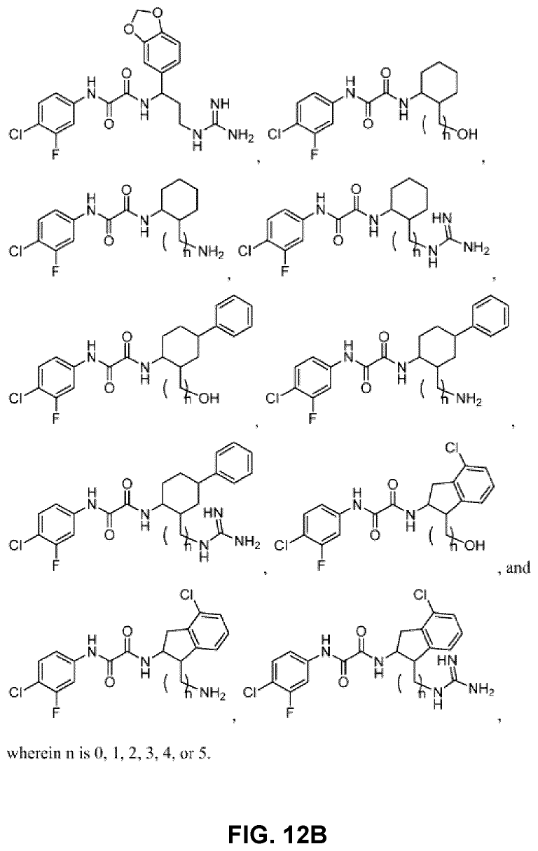

In an embodiment, the gp120 ligand is a small CD4 mimetic (CD4mc). The term “small CD4 mimetic” or “CD4mc” as used herein refers to molecules (e.g., small molecules, peptides, etc.) that bind in the Phe-43 cavity of gp120 and promote the transition of the Env protein to the “open”, CD4-bound conformation. Several CD4mc are known in the art and include, for example, NBD-556, NBD-557, DMJ-I-228, JP-III-48, M48U1 and BNM-III-170. CD4mc are also disclosed in PCT publication No. WO2013/090696 (see FIGS. 12A and B for representative CD4mc compounds), and PCT publication No. WO/2020/028482 (see FIGS. 10A-K for representative CD4mc compounds).

Methods of determining if a HIV-1 Env trimer is in the prefusion closed state 1 conformation include (but are not limited to) negative stain cryogenic electron microscopy, smFRET (Munro et al., Science 2014, 346(6210):759-63) and antibody binding assays using a prefusion mature closed conformation specific antibody, such as VRC26, PGT128, PG9, PGT145, and derivatives thereof, which are well known in the art. Methods of determining if a HIV-1 Env ectodomain trimer is in the CD4-bound open state 2/3 conformation are also provided herein, and include (but are not limited to) negative stain cryogenic electron microscopy and antibody binding assays using a CD4-bound open conformation specific antibody, such as 17b or 19b (available, e.g., from the NIH AIDS Reagent Program, Cat. Nos. 4091 and 11436) which binds to a CD4-induced epitope.

In an embodiment, the agent that induces a state 1 configuration is a conformational blocker.

In an embodiment, the agent that induces a state 1 configuration is one of the HIV fusion inhibitors disclosed in Herschhorn et al., Nat Chem Biol. 2014; 10(10): 845-852, for example one of the following compounds:

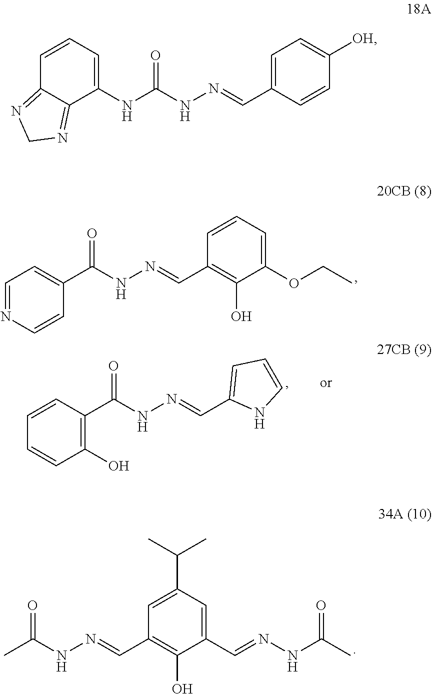

In a further embodiment, the agent is compound 18A.

In an embodiment, the agent that induces a state 1 configuration is one of the HIV fusion inhibitors disclosed in Herschhorn et al., Nat Commun. 2017; 8: 1049, for example one of the following compounds:

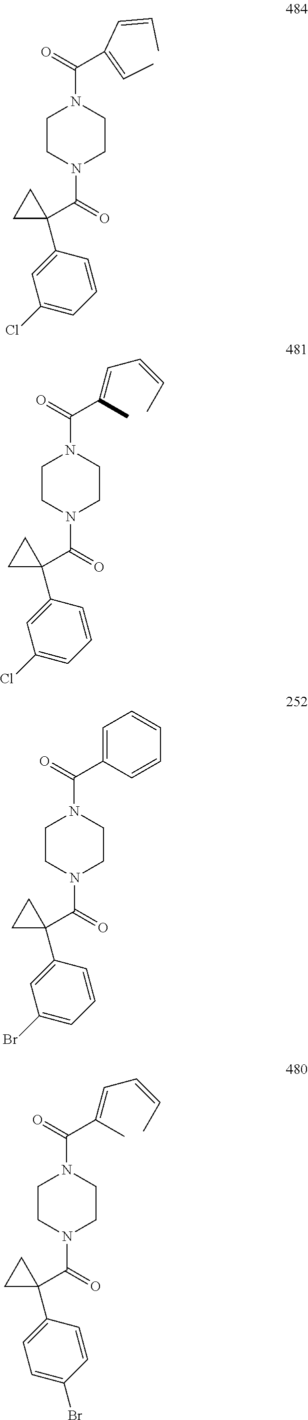

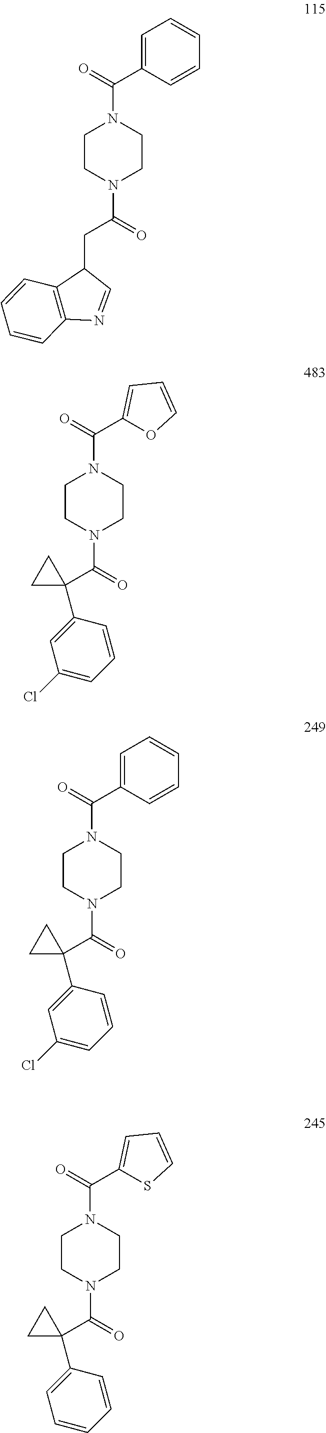 [SMILES: O=C(CC1C=Nc2ccccc21)N1CCN(C(=O)c2ccccc2)CC1 ; O=C(c1ccco1)N1CCN(C(=O)C2(c3cccc(Cl)c3)CC2)CC1 ; O=C(c1ccccc1)N1CCN(C(=O)C2(c3cccc(Cl)c3)CC2)CC1 ; O=C(c1cccs1)N1CCN(C(=O)C2(c3ccccc3)CC2)CC1]

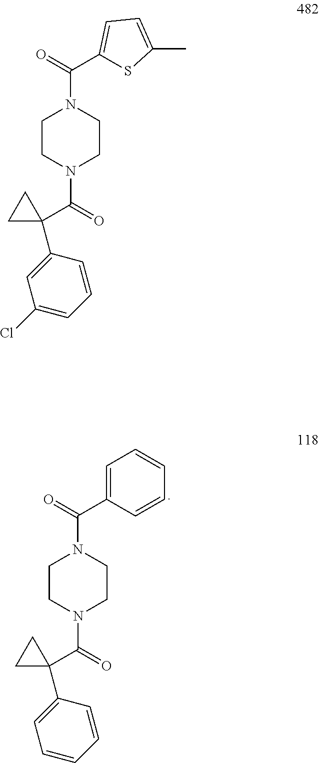 [SMILES: Cc1ccc(C(=O)N2CCN(C(=O)C3(c4cccc(Cl)c4)CC3)CC2)s1 ; O=C(c1ccccc1)N1CCN(C(=O)C2(c3ccccc3)CC2)CC1]

In a further embodiment, the agent is compound 484.

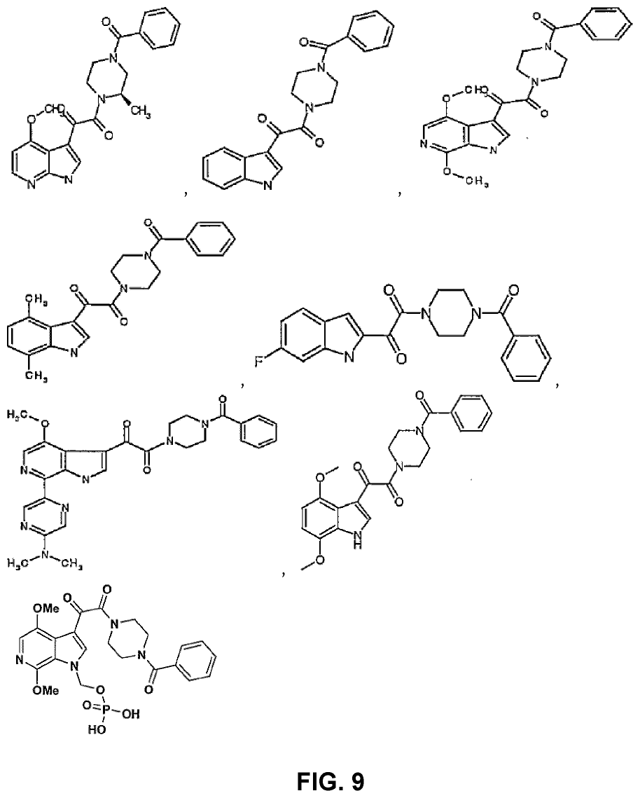

In another embodiment, the agent is one of the compounds disclosed in U.S. Pat. Nos. 7,745,625, 8,168,615, 8,461,333 and 8,871,771 and in PCT publication No. WO 2005/090367. Representative examples of such compounds are depicted in FIG. 9.

In a further embodiment, the agent is temsavir (BMS-626529)

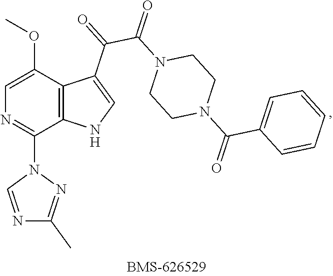 [SMILES: COc1cnc(-n2cnc(C)n2)c2[nH]cc(C(=O)C(=O)N3CCN(C(=O)c4ccccc4)CC3)c12]

or its prodrug fostemsavir (BMS-663068)

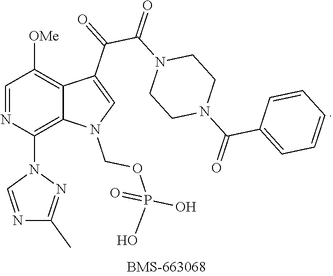 [SMILES: COc1cnc(-n2cnc(C)n2)c2c1c(C(=O)C(=O)N1CCN(C(=O)c3ccccc3)CC1)cn2COP(=O)(O)O]

In an embodiment, the composition further comprises a carrier or excipient, in a further embodiment a pharmaceutically acceptable carrier or excipient. Such compositions may be prepared in a manner well known in the pharmaceutical art by mixing the antibody or an antigen-binding fragment thereof having a suitable degree of purity with one or more optional pharmaceutically acceptable carriers or excipients (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). The carrier/excipient can be suitable for administration of the antibody or an antigen-binding fragment thereof by any conventional administration route, for example, for oral, intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration.

An “excipient” as used herein has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. In certain embodiments, one or more formulations of the dosage form include excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive. Examples of commonly used excipient include water, saline, phosphate buffered saline, dextrose, glycerol, ethanol, and the like, as well as combinations thereof. In many cases, it will be preferable to include isotonic agents, for example, sugars, polyalcohols, such as mannitol, sorbitol, or sodium chloride in the composition. Additional examples of pharmaceutically acceptable substances are wetting agents or auxiliary substances, such as emulsifying agents, preservatives, or buffers, which increase the shelf life or effectiveness.

In an embodiment, the composition further comprises a vaccine adjuvant. The term “vaccine adjuvant” refers to a substance which, when added to an immunogenic agent such as an antigen, non-specifically enhances or potentiates an immune response to the agent in the host upon exposure to the mixture. Suitable vaccine adjuvants are well known in the art and include, for example: (1) mineral salts (aluminum salts such as aluminum phosphate and aluminum hydroxide, calcium phosphate gels), squalene, (2) oil-based adjuvants such as oil emulsions and surfactant based formulations, e.g., incomplete or complete Freud's adjuvant, MF59 (microfluidised detergent stabilised oil-in-water emulsion), QS21 (purified saponin), AS02 [SBAS2] (oil-in-water emulsion+MPL+QS-21), (3) particulate adjuvants, e.g., virosomes (unilamellar liposomal vehicles incorporating influenza haemagglutinin), AS04 ([SBAS4] aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL+M. Phlei cell wall skeleton), AGP [RC-529] (synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified LT and CT (genetically modified bacterial toxins to provide non-toxic adjuvant effects), complete Freud's adjuvant (comprising inactivated and dried mycobacteria) (5) endogenous human immunomodulators, e.g., hGM-CSF or hIL-12 (cytokines that can be administered either as protein or plasmid encoded), Immudaptin (C3d tandem array) and/or (6) inert vehicles, such as gold particles.

In an embodiment, the mutated HIV-1 gp120 polypeptide or composition may be comprised in a cell, a liposome or a virus-like particle (VLP). Thus, in another aspect, the present disclosure provides a cell, liposomes (see, e.g., Rao et al., J Infect Dis. 2018; 218(10):1541-1550) or VLP expressing at its surface the mutated HIV-1 gp120 polypeptide disclosed herein, for example in the form of a trimer with gp41. VLPs are multimeric nanostructures morphologically resembling authentic viral particles composed of viral structural proteins with inherent self-assembly properties but are devoid of viral genetic materials. The display of HIV Env trimers at the surface of VLPs is considered a promising strategy for eliciting an immune response (e.g., neutralizing antibodies) against HIV (Zhao et al., Vaccines (Basel). 2016; 4(1): 2).

In an embodiment, the mutated HIV-1 gp120 polypeptide may be delivered in the form of a nucleic acid comprising a sequence encoding the mutated HIV-1 gp120 polypeptide. The nucleic acid may be optimized, such as by codon optimization, for expression in a targeted mammalian subject (e.g., human). As discussed below, the nucleic acid may be incorporated into a vector (e.g., a viral vector, such as an adenovirus or poxvirus vector). Accordingly, the composition or vaccine disclosed herein may include one or more of these vectors. The mutated HIV-1 gp120 polypeptide may be recombinantly expressed in a cell or organism, or may be directly administered to a subject (e.g., a human) infected with, or at risk of becoming infected with, HIV (e.g., HIV-1).

The present disclosure also provides vectors including the nucleic acid molecule encoding the mutated HIV-1 gp120 polypeptide. The vector can be, for example, a carrier (e.g., a liposome), a plasmid, a cosmid, a yeast artificial chromosome, or a virus (e.g., an adenovirus vector or a poxvirus vector) that comprises the nucleic acid molecule encoding the mutated HIV-1 gp120 polypeptide.

The adenovirus vector may be derived from a recombinant adenovirus serotype 11 (Ad11), adenovirus serotype 15 (Ad15), adenovirus serotype 24 (Ad24), adenovirus serotype 26 (Ad26), adenovirus serotype 34 (Ad34), adenovirus serotype 35 (Ad35), adenovirus serotype 48 (Ad48), adenovirus serotype 49 (Ad49), adenovirus serotype 50 (Ad50), Pan9 (AdC68), or a chimeric variant thereof (e.g., adenovirus serotype 5 HVR48 (Ad5HVR48)). The poxvirus vector may be derived, for example, from modified vaccinia virus Ankara (MVA). These vectors can include additional nucleic acid sequences from several sources.

Such vectors may be constructed using any recombinant molecular biology technique known in the art. The vector, upon transfection or transduction of a target cell or organism, can be extrachromosomal or integrated into the host cell chromosome. The nucleic acid component of a vector can be in single or multiple copy number per target cell, and can be linear, circular, or concatamerized. The vectors can also include internal ribosome entry site (IRES) sequences to allow for the expression of multiple peptide or polypeptide chains from a single nucleic acid transcript (e.g., a polycistronic vector, e.g., a bi- or tri-cistronic vector).

Vectors may also include gene expression elements that facilitate the expression of the encoded mutated HIV-1 gp120 polypeptide. Gene expression elements include, but are not limited to, (a) regulatory sequences, such as viral transcription promoters and their enhancer elements, such as the SV40 early promoter, Rous sarcoma virus LTR, and Moloney murine leukemia virus LTR; (b) splice regions and polyadenylation sites such as those derived from the SV40 late region; and (c) polyadenylation sites such as in SV40. Also included are plasmid origins of replication, antibiotic resistance or selection genes, multiple cloning sites (e.g., restriction enzyme cleavage loci), and other viral gene sequences (e.g., sequences encoding viral structural, functional, or regulatory elements, such as the HIV long terminal repeat (LTR)).

To improve the delivery of the nucleic acid into a cell or subject in order to promote formation of the Env trimers, lipoplexes (e.g., liposomes) and polyplexes can be used to protect the nucleic acid from undesirable degradation during the transfection process. The nucleic acid molecules can be covered with lipids (e.g., cationic lipids) in an organized structure like a micelle or a liposome. When the organized structure is complexed with the nucleic acid molecule it is called a lipoplex. Cationic lipids, due to their positive charge, naturally complex with the negatively-charged nucleic acid, and are thus preferred for such liposomes. Polyplexes refer to complexes of polymers with nucleic acids.

Exemplary cationic lipids and polymers that can be used in combination with one or more of the nucleic acid molecules encoding mutated HIV-1 gp120 polypeptide to form lipoplexes or polyplexes include, but are not limited to, polyethylenimine, lipofectin, lipofectamine, polylysine, chitosan, trimethylchitosan, and alginate.

In another aspect, the present disclosure provides a method for eliciting an immune response to HIV-1 in a subject, comprising administering to the subject a prophylactically or therapeutically effective amount of (i) the mutated HIV-1 gp120 polypeptide defined herein, and (ii) a gp120 or gp41 ligand. In an embodiment, the composition defined herein is administered.

In another aspect, the present disclosure provides the use of (i) the mutated HIV-1 gp120 polypeptide defined herein, and (ii) a gp120 or gp41 ligand, for eliciting an immune response to HIV-1 in a subject. In an embodiment, the composition defined herein is used.

In another aspect, the present disclosure provides the use of (i) the mutated HIV-1 gp120 polypeptide defined herein, and (ii) a gp120 or gp41 ligand, for the manufacture of a medicament for eliciting an immune response to HIV-1 in a subject. In an embodiment, the composition defined herein is used.

In another aspect, the present disclosure provides a combination comprising (i) the mutated HIV-1 gp120 polypeptide defined herein, and (ii) a gp120 or gp41 ligand for eliciting an immune response to HIV-1 in a subject. In an embodiment, the combination is present in the composition defined herein.

When treating disease (e.g., HIV infection/AIDS), the mutated HIV-1 gp120 polypeptide and gp120 or gp41 ligand, combination or composition disclosed herein may be administered to the subject either before the occurrence of symptoms or a definitive diagnosis or after diagnosis or symptoms become evident. For example, the composition may be administered, for example, immediately after diagnosis or the clinical recognition of symptoms or 2, 4, 6, 10, 15, or 24 hours, 2, 3, 5, or 7 days, 2, 4, 6 or 8 weeks, or even 3, 4, or 6 months after diagnosis or detection of symptoms. In an embodiment, the mutated HIV-1 gp120 polypeptide and gp120 or gp41 ligand, combination or composition disclosed herein is administered to a subject that is not infected by HIV, e.g., as a prophylactic vaccine to confer immune protection (partial or complete) against future HIV-1 infections, for example a subject at-risk of being infected. In an embodiment, the mutated HIV-1 gp120 polypeptide and gp120 or gp41 ligand, combination or composition disclosed herein is administered to a subject that is already infected by HIV, e.g., as a therapeutic vaccine to boost the immune response against HIV-1 and reduce viral load.

The mutated HIV-1 gp120 polypeptide and gp120 or gp41 ligand, combination or composition disclosed herein may be administered in combination with one or more additional therapeutic agents, for example, for preventing or treating an HIV infection (e.g., an HIV-1 infection) in a subject. Such additional therapeutic agents can include, for example, a broadly neutralizing antibody (bnAb), e.g., those described in PCT publications No. WO2015/048770, WO 2012/030904, and WO 2013/055908. Exemplary bnAbs that can be administered in combination with the compositions of the invention include PGT121, PGT122, PGT123, PGT124, PGT125, PGT126, PGT127, PGT128, PGT130, PGT131, PGT132, PGT133, PGT134, PGT135, PGT136, PGT137, PGT138, PGT139, PGT141, PGT142, PGT143, PGT144, PGT145, PGT151, PGT152, PGT153, PGT154, PGT155, PGT156, PGT157, PGT158, 3BNC117 and 10-1074, a derivative or clonal relative thereof, or a combination thereof.

The additional therapeutic agent may also be an antiretroviral therapy (ART), which may, e.g., be selected from any one or more of the following, or combinations thereof: efavirenz, emtricitabine, and tenofovir disoproxil fumarate (Atripla); emtricitabine, rilpivirine, and tenofovir disoproxil fumarate (Complera); elvitegravir, cobicistat, emtricitabine, and tenofovir disoproxil fumarate (Stribild); lamivudine and zidovudine (Combivir); emtricitabine, FTC (Emtriva); lamivudine, 3TC (Epivir); abacavir and lamivudine (Ebzicom); zalcitabine, dideoxycytidine, ddC (Hivid); zidovudine, azidothymidine, AZT, ZDV (Retrovir); abacavir, zidovudine, and lamivudine (Trizivir); tenofovir disoproxil fumarate and emtricitabine (Truvada); enteric coated didanosine, ddl EC (Videx EC); didanosine, dideoxyinosine, ddl (Videx); tenofovir disoproxil fumarate, TDF (Viread); stavudine, d4T (Zerit); abacavir sulfate, ABC (Ziagen); Rilpivirine (Edurant); Etravirine (Intelence); delavirdine, DLV (Rescriptor); efavirenz, EFV (Sustiva); nevirapine, NVP (Viramune or Viramune XR); amprenavir, APV (Agenerase); tipranavir, TPV (Aptivus); indinavir, IDV (Crixivan); saquinavir (Fortovase); saquinavir mesylate, SQV (Invirase); lopinavir and ritonavir, LPV/RTV (Kaletra); Fosamprenavir Calcium, FOS-APV (Lexiva); ritonavir, RTV (Norvir); Darunavir (Prezista); atazanavir sulfate, ATV (Reyataz); nelfinavir mesylate, NFV (Viracept); enfuvirtide, T-20 (Fuzeon); maraviroc (Selzentry); raltegravir, RAL (Isentress); and dolutegravir (Tivicay).

The additional therapeutic agent can also be an immunomodulator. The immunomodulator may be selected, e.g., from any one or more of the following, or combinations thereof: AS-101, Bropirimine, Acemannan, CL246,738, EL10, FP-21399, Gamma Interferon, Granulocyte Macrophage Colony Stimulating Factor, HIV Core Particle Immunostimulant, IL-2, Immune Globulin Intravenous, IMREG-1, IMREG-2, Imuthiol Diethyl Dithio Carbamate, Alpha-2 Interferon, Methionine-Enkephalin, MTP-PE, Muramyl-Tripeptide, Granulocyte Colony Stimulating Factor, Remune, CD4 (e.g., recombinant soluble CD4), rCD4-IgG hybrids, SK&F106528 Soluble T4, Thymopentin, Tumor Necrosis Factor, and Infliximab.

The additional therapeutic agent can also be a reservoir activator. The reservoir activator may be selected, e.g., from any one or more of the following, or combinations thereof: histone deacytelase (HDAC) inhibitors (e.g., romidepsin, vorinostat, and panobinostat), immunologic activators (e.g., cytokines and TLR agonists), and dedicated small molecule drugs.

Administration of an additional therapeutic agent may be prior to, concurrent with, or subsequent to the administration of the composition or vaccine disclosed herein.

In another aspect, the present disclosure relates to a method for determining whether a test agent (e.g., an antibody) binds to an HIV Env trimer into an open (state 2/3) conformation comprising contacting said test agent with the mutated HIV-1 gp120 polypeptide defined herein, and a gp120 ligand capable of inducing an open (state 2/3) conformation, e.g., a CD4mc.

In another aspect, the present disclosure relates to a method (e.g., in vitro) for determining whether a test agent (e.g., an antibody) binds to an HIV Env trimer into a closed (state 1) conformation comprising contacting said test agent with the mutated HIV-1 gp120 polypeptide defined herein, and a gp120 ligand capable of inducing a closed (state 1) conformation, e.g., a conformational blocker.

In another aspect, the present disclosure relates to a method (e.g., in vitro) for inducing an HIV Env trimer into an open (state 2/3) conformation comprising contacting an HIV Env trimer comprising the mutated HIV-1 gp120 polypeptide defined herein with a gp120 ligand capable of inducing an open (state 2/3) conformation, e.g., a CD4mc.

In another aspect, the present disclosure relates to the use of the mutated HIV-1 gp120 polypeptide defined herein with a gp120 ligand capable of inducing an open (state 2/3) conformation, e.g., a CD4mc, for inducing an HIV Env trimer into an open (state 2/3) conformation, or for the manufacture of a medicament for inducing an HIV Env trimer into an open (state 2/3) conformation.

In another aspect, the present disclosure relates to a method (e.g., in vitro) for inducing an HIV Env trimer into a closed (state 1) conformation comprising contacting an HIV Env trimer comprising the mutated HIV-1 gp120 polypeptide defined herein with a gp120 ligand capable of inducing a closed (state 1) conformation, e.g., a conformational blocker.

In another aspect, the present disclosure relates to the use of the mutated HIV-1 gp120 polypeptide defined herein with a gp120 ligand capable of inducing a closed (state 1) conformation, e.g., a conformational blocker, for inducing an HIV Env trimer into a closed (state 1) conformation, or for the manufacture of a medicament for inducing an HIV Env trimer into a closed (state 1) conformation.

In another aspect, the present disclosure relates to a method for determining whether a test agent induces a closed (state 1) conformation of an HIV Env trimer comprising (a) contacting the mutated HIV-1 gp120 polypeptide defined herein with said test agent, and (b) determining whether the HIV Env trimer is in a closed (state 1) conformation.

In another aspect, the present disclosure relates to a method for determining whether a test agent induces an open (state 2/3) conformation of an HIV Env trimer comprising (a) contacting the mutated HIV-1 gp120 polypeptide defined herein with said test agent, and (b) determining whether the HIV Env trimer is in an open (state 2/3) conformation.

Such determining may be performed using assays capable of measuring conformational changes of membrane-bound trimeric Env, for example, antibodies that specifically binds to the closed (state 1) or open (state 2/3) conformation, as described above, or the assay described in Veillette M et al., 2014. J Vis Exp doi:10.3791/51995:51995 or Haim H et al., PLoS Pathog 7:e1002101.

### MODE(S) FOR CARRYING OUT THE INVENTION

The present invention is illustrated in further details by the following non-limiting examples.

### Example 1: Comparison of Phe43 Cavity and Co-Evolving Inner Domain Layers Residues Among HIV-1 Strains

All HIV-1 sequences have been analyzed together or segregated by clades using the NIH Los Alamos HIV database to determine the degree of conservation of residues located in layer 1 (residue 61), layer 2 (residues 105 and 108), layer 3 (residues 474, 475 and 476) (collectively named LM for layer mutants) and the Phe43 cavity residue 375. By comparing consensus sequences from CRF01_AE strains with the different HIV-1 group M clades (clades A to K), it is possible to see the divergence between CRF01_AE and all other clades (FIG. 1A). Among the coevolving inner domain residues, most of the clades share the same consensus sequence (Y61, H105, 1108, D474, M475, R476) except for clade F (N474 and K476) and clade J (K476), which differ from residues found in CRF01_AE strains (H/Q61, Q105, V108, N474, 1475, K/R476). Serine 375 is the predominant residue (>75%) in all HIV-1 major clades, except for clade K, and CRF01_AE (FIGS. 1A-C). Other residues can occupy position 375 (T375, N375, 1375, M375) with T375 being present in more than 8% of strains (FIG. 1B). T375 is present in clade B (16,9%), clade A1 (5%) and clade C (4,87%). Interestingly, CRF01_AE strains have a highly conserved histidine at position 375 (H375, >99%) (Zoubchenok D et al., 2017. J Virol 91; Prevost J. et al., 2017. J Virol 91).

### Example 2: Effect of Gp120 Layer Mutations (LM) on Neutralization by Soluble CD4 and CD4-Mimetic Compounds

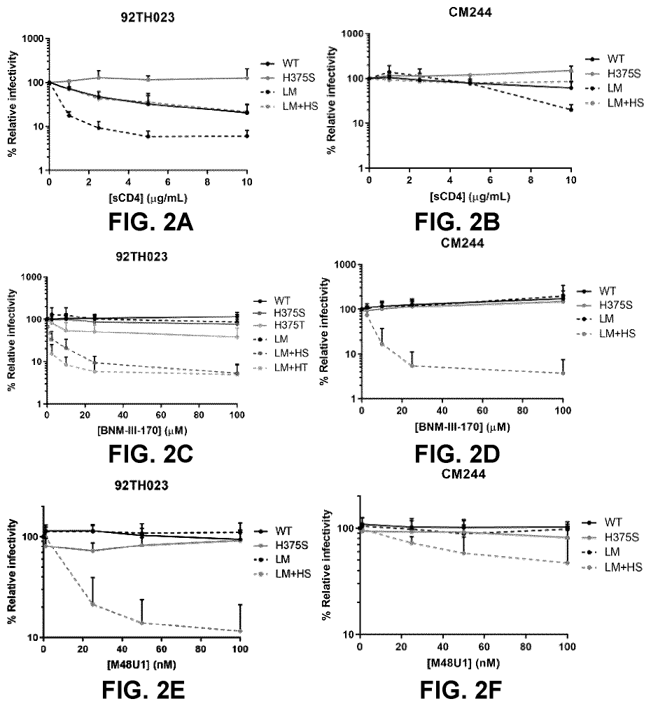

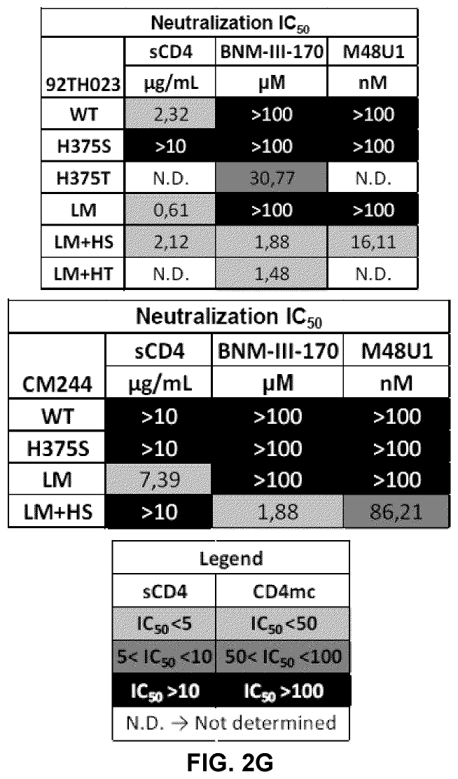

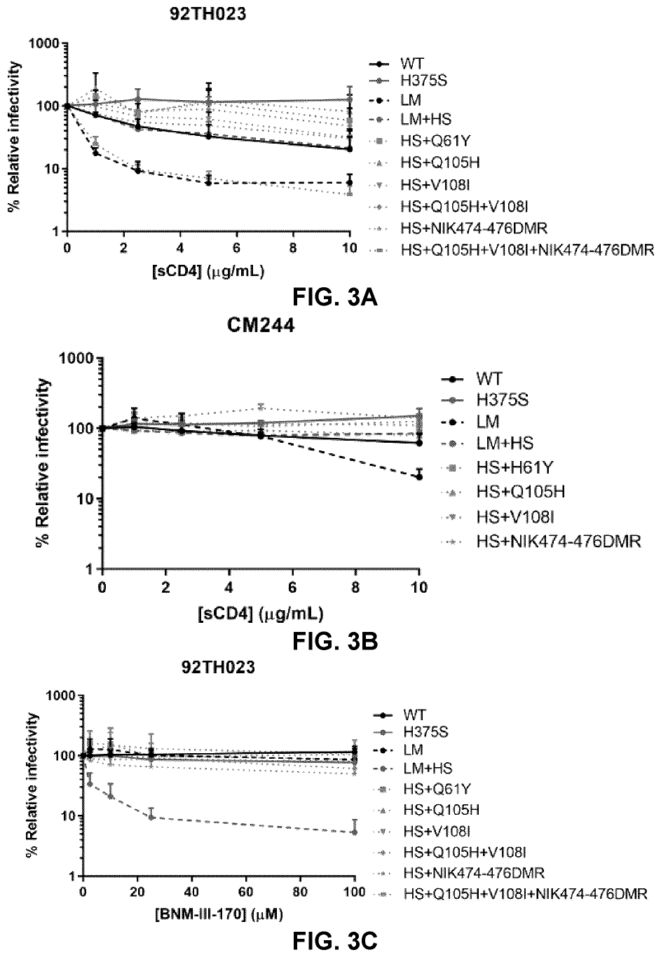

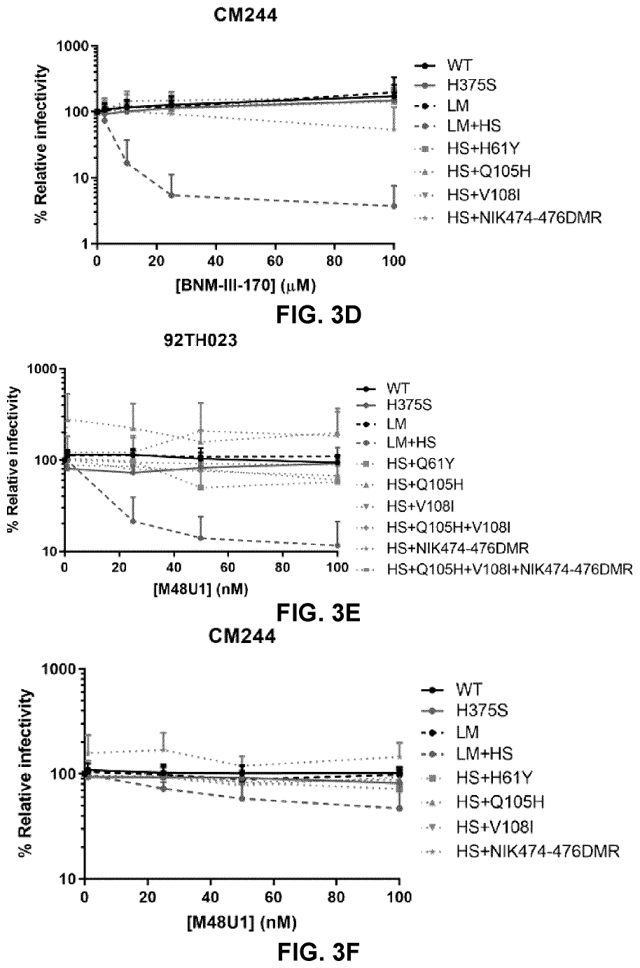

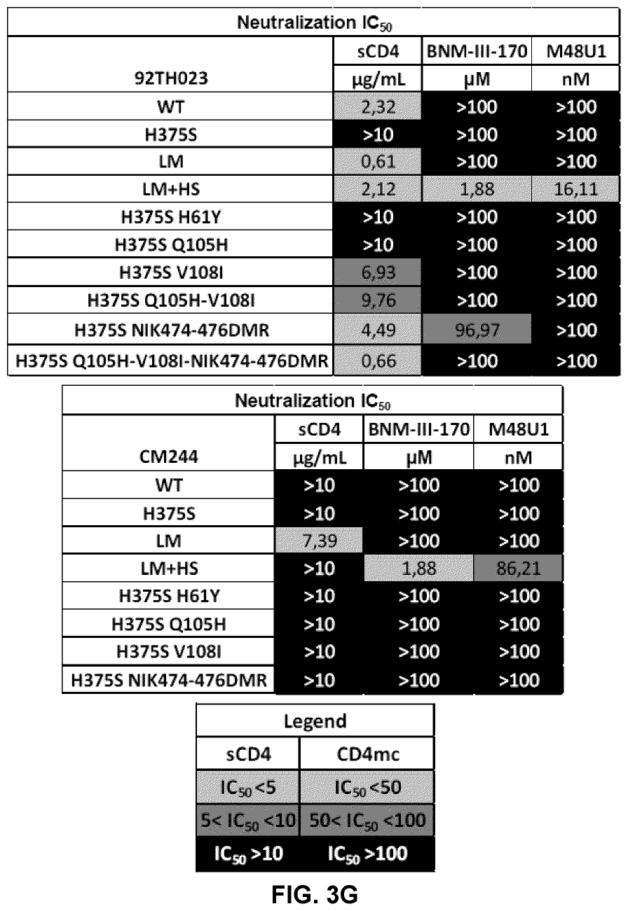

CD4mc were used as probes to evaluate the potential impact of the LM residues on shaping the Phe43 cavity. First, the effect of the H375S mutation on the sensitivity of two CRF01_AE isolates (tier 1 92TH023 and tier 2 CM244) to neutralization by different CD4 mimics including soluble CD4 (sCD4), CD4mc (BNM-III-170) and a CD4 miniprotein (M48U1) was assessed. Replacement of histidine by a serine at position 375 (H375S) into both HIV-1_{CRF01_AE} Envs completely abolished the susceptibility of pseudotypes tosCD4 neutralization (FIGS. 2A-B,G). Viral particles bearing both CRF01_AE Envs were resistant to neutralization by BNM-III-170 and M48U1. Interestingly, replacement of the bulky histidine by a serine at this position (H375S) did not restore neutralization sensibility to these CD4 mimics (FIGS. 2C-G). However, this change introduced in combination with the LM mutations (LM+HS) dramatically enhanced the susceptibility of both CRF01_AE strains to neutralization by both CD4 mimics. Of note, different combinations of single or multiple layers mutations together with the H375S mutation did not restore sensitivity to BNM-III-170 or M48U1 neutralization (FIG. 3). This was different from sCD4 neutralization, where LM mutations without the Q61Y change was also sensitive to sCD4 neutralization (FIG. 3), thus highlighting subtle differences in their mode of recognition of Env. Altogether, these results indicate that the presence of the H375S (HS) or H375T (HT) mutations in the Phe43 cavity combined with the inner domain layer mutations (LM) CRF01_AE Envs from 92TH023 and CM244 isolates renders the recombinant HIV more sensitive to neutralization by soluble CD4 (sCD4) and CD4-mimetic (CD4mc) compounds (small CD4mc BNM-III-170 and CD4mc peptide M48U1). In contrast, no or very little effect was measured in recombinant viruses with mutations in the Phe43 cavity only or in the inner domain layer only.

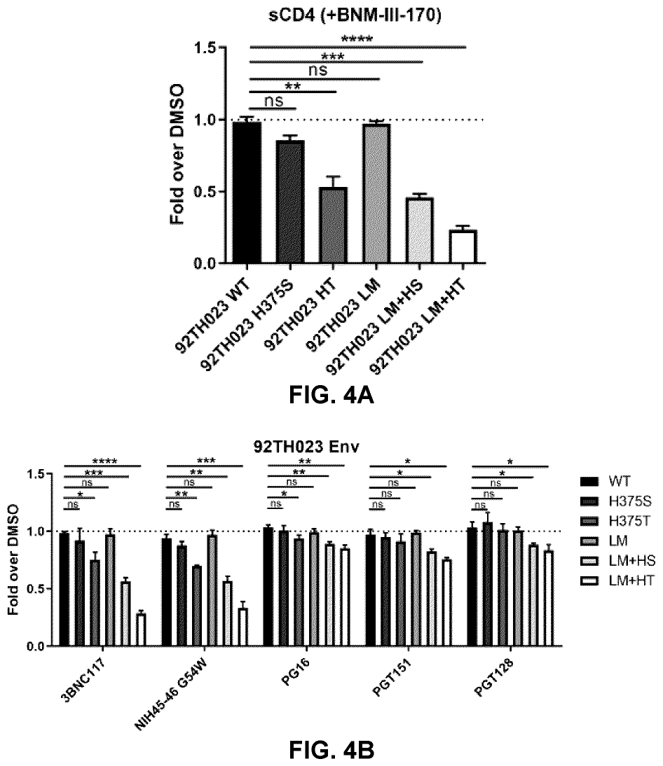

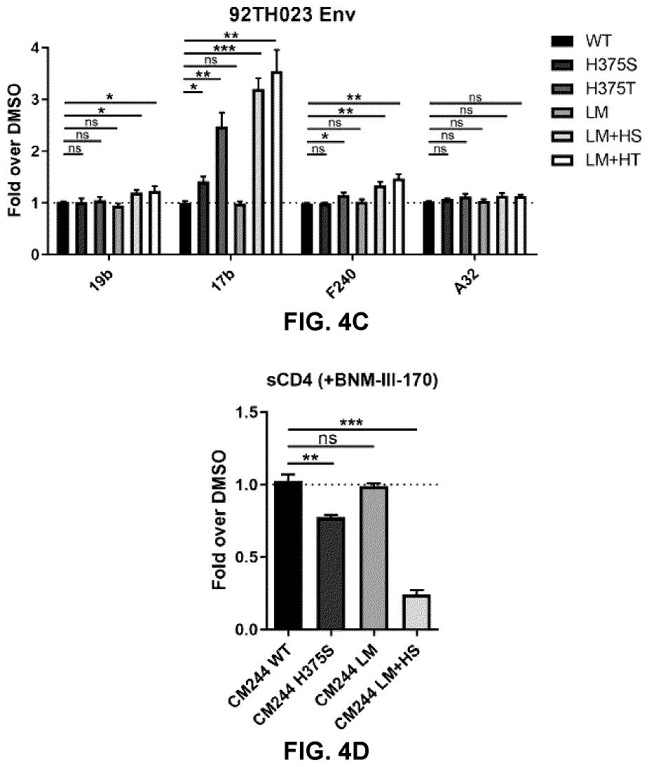

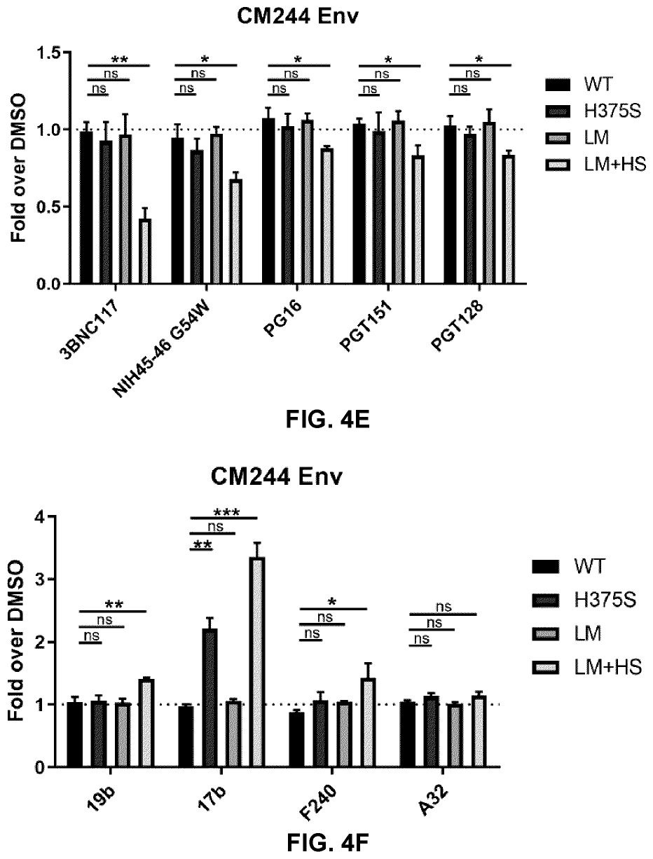

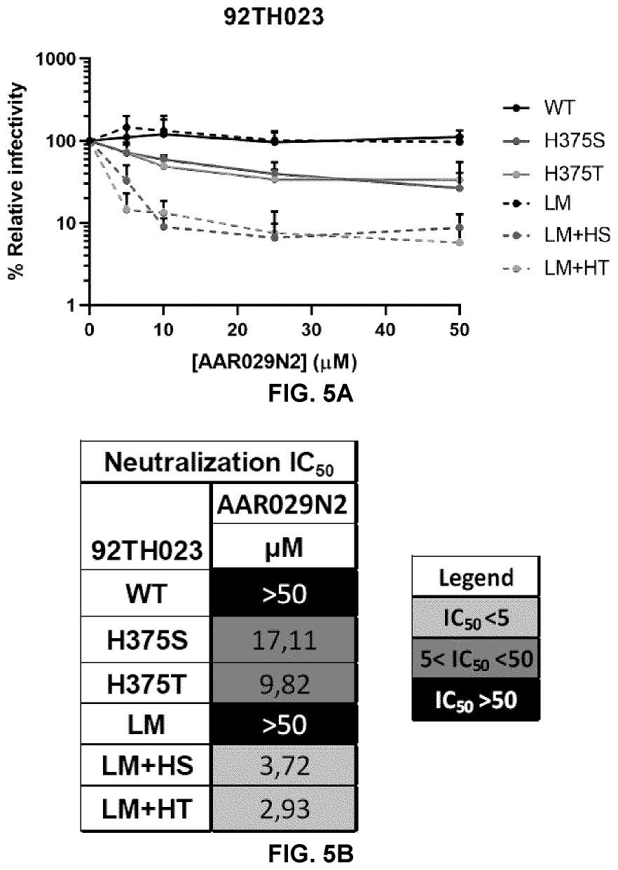

To gain a better understanding of the impact that the LM and 375 changes have on Env conformation, an assay to measure sCD4 and BNM-III-170 capacity to interact with Env was developed. Briefly, CD4-negative cells were transfected with Env variants. Two days post-transfection, BNM-III-170 or the vehicle (DMSO) was added to Env-expressing cells. The impact on Env conformation was detected by evaluating binding of broadly-neutralizing antibodies (bNAbs) that preferentially recognize the “closed” state 1 trimer (3BNC117, NIH45-46 G54W, PG16, PGT121 and PGT128), non-neutralizing (nnAbs) CD4i Abs (17b, 19b, F240 and A32) or soluble CD4 (sCD4) that preferentially recognize the “open” state 2/3 Env conformation (Munro J B, et al., 2014. Science 346:759-763; Lu M, et al. 2019. Nature 568:415-419; Derking R. et al. 2015. PLoS Pathog 11: e1004767; Ma X. et al. 2018, Elife 7; Alsahafi N. et al. 2019. Cell Host Microbe 25:578-587 e575). Their ability to interact with Env was obtained by calculating the decrease of these ligands binding compared to DMSO. Overall, the results presented in FIG. 4 indicate that the combination of LM+HS or LM+HT is superior in adopting an “open” Env conformation in presence of the CD4mc BNM-III-170 as shown by a decrease in recognition of state 1 preferring bNAbs, and an increase in sCD4 and nnAbs binding that preferentially recognize the “open” state 2/3 Env conformation. Interestingly, the superior capacity of LM+HS or LM+HT mutants to respond to Env inhibitor was supported by their enhanced susceptibility to a new class of Env inhibitors: cyclic peptide triazoles (cPT) AAR029N2 (15) (FIG. 5).

### Example 3: Phe43 Cavity and Layers Mutations Render CRF01_AE Strains Susceptible to Conformational Blockers and CD4-Binding Site Antibodies

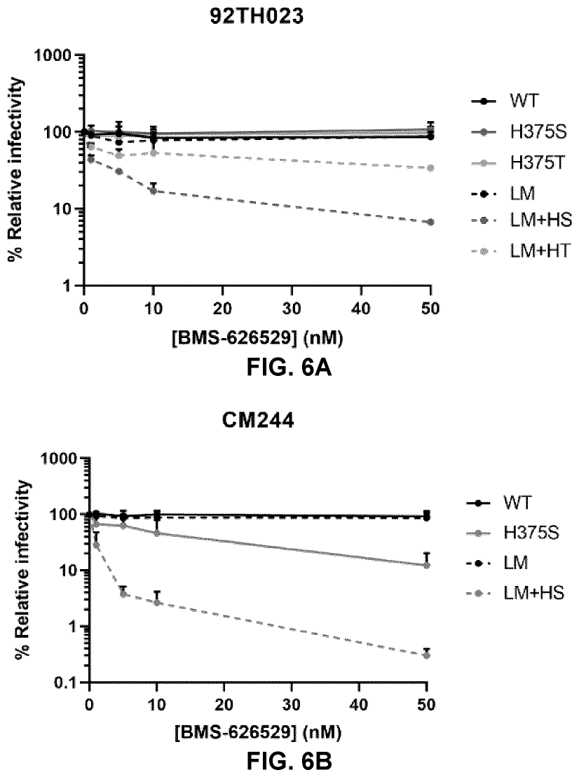

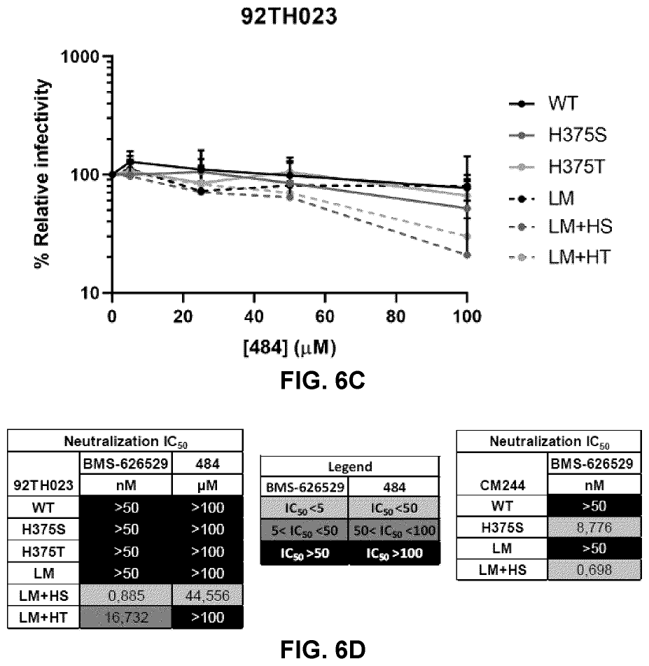

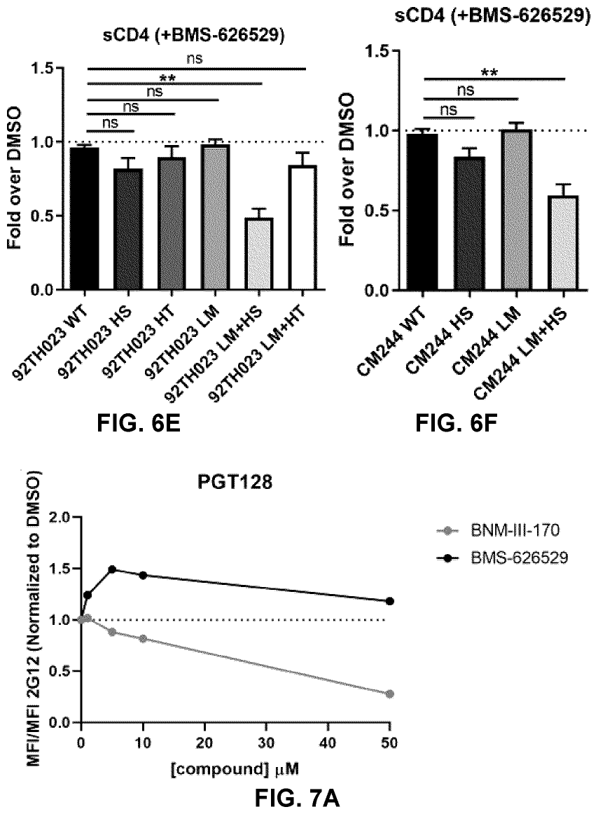

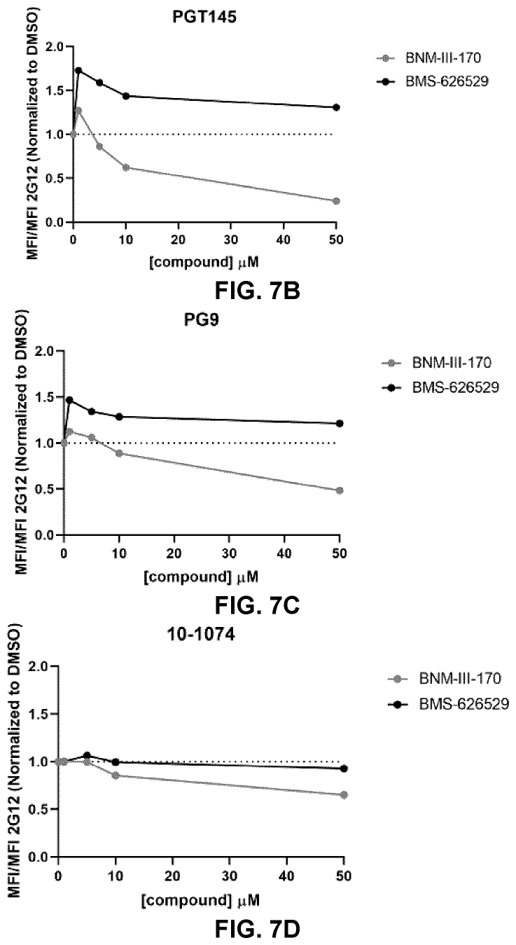

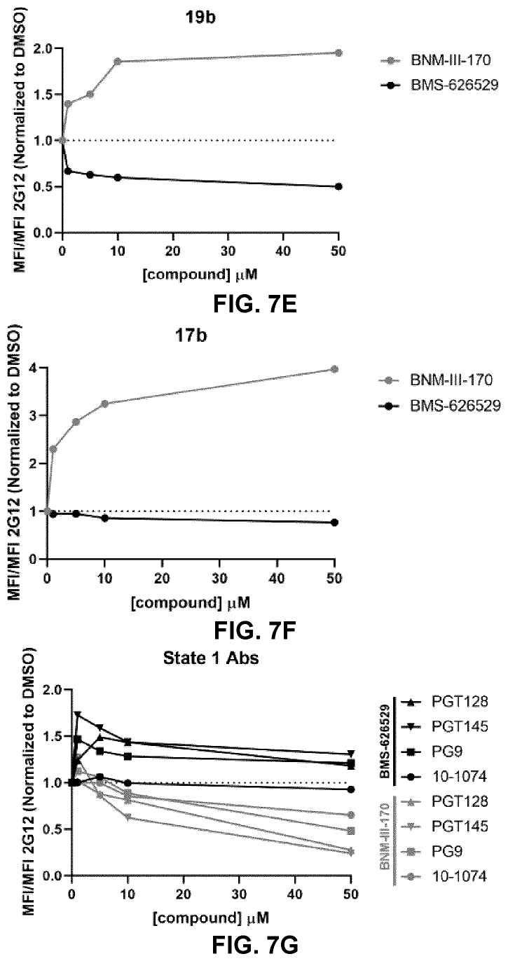

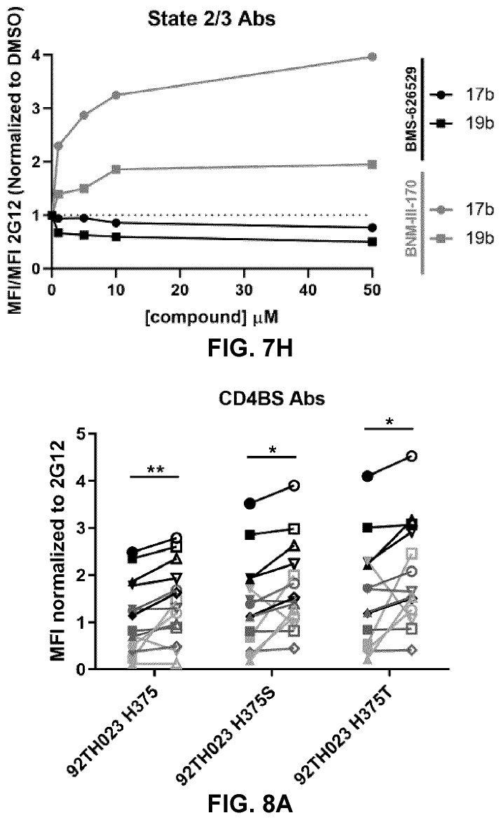

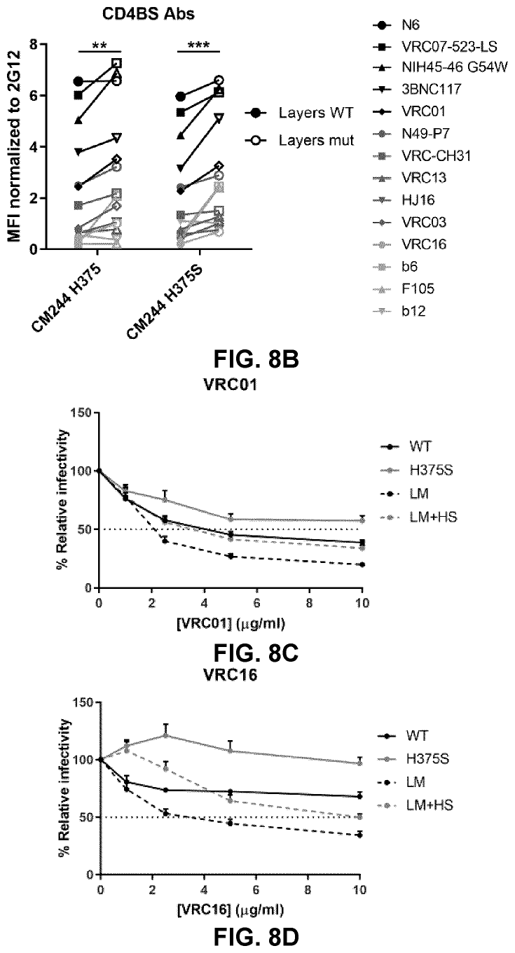

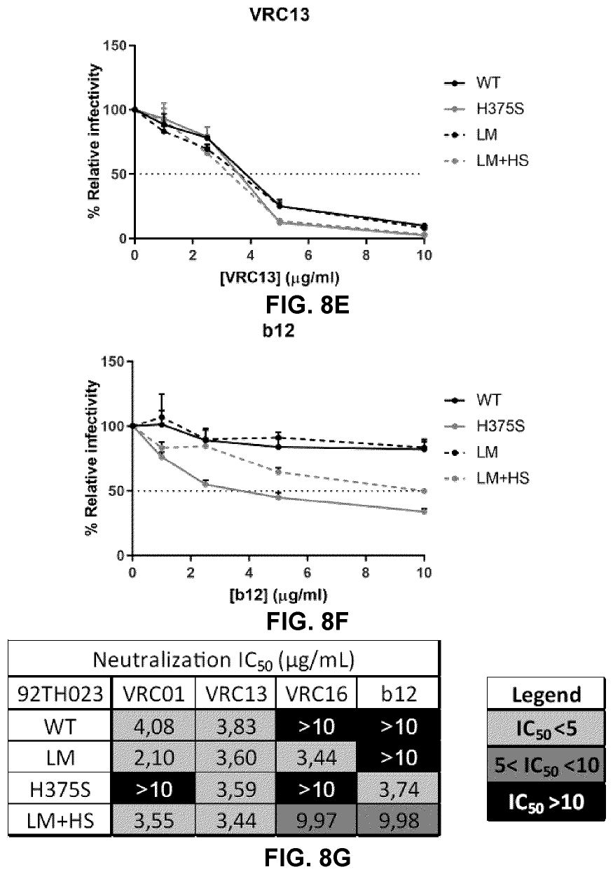

To assess whether the LM+HS or LM+HT mutations were restricted to inhibitors “pushing” Env to more “open” (state 2/3) conformations or if they could enhance Env flexibility in such a way that they could respond to inhibitors “pushing” in opposite directions (to a closed, state 1 conformations), their susceptibility to conformational blockers (BMS-626529 and 484) known to stabilize Env state 1 (10, 16, 17) was tested. Strikingly, these unique set of mutations sensitized CRF01_AE Env to neutralization to BMS-626529 and 484 (FIG. 6) and enhanced Env recognition by bNAbs preferentially binding state 1 in presence of the conformational blocker BMS-626529, or by nnAbs that preferentially bind to Env in states 2/3 in presence of the CD4mc BNM-III-170 (FIG. 7). Finally, it was observed that the LM+HS or LM+HT combination significantly enhanced recognition of Env by CD4-binding site (CD4BS) antibodies (FIGS. 8A-B), which was consistent with enhanced susceptibility of the LM+HS or LM+HT to these antibodies and concomitant increase in their IC₅₀ (FIGS. 8C-G).

Overall, the studies described herein reveal a complex interplay between the gp120 inner domain and the Phe43 cavity that could be exploited to guide the development of more potent Env inhibitors (CD4mc and conformational blockers), help expose the CD4 binding site and the elusive state 1 conformation, all properties highly thought to develop an efficient HIV-1 vaccine.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Allan J S, Coligan J E, Barin F, McLane M F, Sodroski J G, Rosen C A, Heseltine W A, Lee T H, Essex M. 1985. Major glycoprotein antigens that induce antibodies in AIDS patients are encoded by HTLV-III. Science 228:1091-1094.
2. Robey W G, Safai B, Oroszlan S, Arthur L O, Gonda M A, Gallo R C, Fischinger P J. 1985. Characterization of envelope and core structural gene products of HTLV-III with sera from AIDS patients. Science 228:593-595.
3. Wyatt R, Sodroski J. 1998. The HIV-1 envelope glycoproteins: fusogens, antigens, and immunogens. Science 280:1884-1888.
4. Alkhatib G, Combadiere C, Broder C C, Feng Y, Kennedy P E, Murphy P M, Berger E A. 1996. CC CKR5: a RANTES, MIP-1alpha, MIP-1beta receptor as a fusion cofactor for macrophage-tropic HIV-1. Science 272:1955-1958.
5. Choe H, Farzan M, Sun Y, Sullivan N, Rollins B, Ponath P D, Wu L, Mackay C R, LaRosa G, Newman W, Gerard N, Gerard C, Sodroski J. 1996. The beta-chemokine receptors CCR3 and CCR5 facilitate infection by primary HIV-1 isolates. Cell 85:1135-1148.
6. Deng H, Liu R, Ellmeier W, Choe S, Unutmaz D, Burkhart M, Di Marzio P, Marmon S, Sutton R E, Hill C M, Davis C B, Peiper S C, Schell T J, Littman D R, Landau N R. 1996. Identification of a major co-receptor for primary isolates of HIV-1. Nature 381:661-666.
7. Doranz B J, Rucker J, Yi Y, Smyth R J, Samson M, Peiper S C, Parmentier M, Collman R G, Doms R W. 1996. A dual-tropic primary HIV-1 isolate that uses fusin and the beta-chemokine receptors CKR-5, CKR-3, and CKR-2b as fusion cofactors. Cell 85:1149-1158.
8. Dragic T, Litwin V, Allaway G P, Martin S R, Huang Y, Nagashima K A, Cayanan C, Maddon P J, Koup R A, Moore J P, Paxton W A. 1996. HIV-1 entry into CD4+ cells is mediated by the chemokine receptor CC-CKR-5. Nature 381:667-673.
9. Feng Y, Broder C C, Kennedy P E, Berger E A. 1996. HIV-1 entry cofactor: functional cDNA cloning of a seven-transmembrane, G protein-coupled receptor. Science 272:872-877.
10. Wu L, Gerard N P, Wyatt R, Choe H, Parolin C, Ruffing N, Borsetti A, Cardoso A A, Desjardin E, Newman W, Gerard C, Sodroski J. 1996. CD4-induced interaction of primary HIV-1 gp120 glycoproteins with the chemokine receptor CCR-5. Nature 384:179-183.
11. Trkola A, Dragic T, Arthos J, Binley J M, Olson W C, Allaway G P, Cheng-Mayer C, Robinson J, Maddon P J, Moore J P. 1996. CD4-dependent, antibody-sensitive interactions between HIV-1 and its co-receptor CCR-5. Nature 384:184-187.
12. Furuta R A, Wild C T, Weng Y, Weiss C D. 1998. Capture of an early fusion-active conformation of HIV-1 gp41. Nat Struct Biol 5:276-279.
13. He Y, Vassell R, Zaitseva M, Nguyen N, Yang Z, Weng Y, Weiss C D. 2003. Peptides trap the human immunodeficiency virus type 1 envelope glycoprotein fusion intermediate at two sites. J Virol 77:1666-1671.
14. Koshiba T, Chan D C. 2003. The prefusogenic intermediate of HIV-1 gp41 contains exposed C-peptide regions. J Biol Chem 278:7573-7579.
15. Kwong P D, Wyatt R, Robinson J, Sweet R W, Sodroski J, Hendrickson W A. 1998. Structure of an HIV gp120 envelope glycoprotein in complex with the CD4 receptor and a neutralizing human antibody. Nature 393:648-659.
16. Veillette M, Desormeaux A, Medjahed H, Gharsallah N E, Coutu M, Baalwa J, Guan Y, Lewis G, Ferrari G, Hahn B H, Haynes B F, Robinson J E, Kaufmann D E, Bonsignori M, Sodroski J, Finzi A. 2014. Interaction with cellular CD4 exposes HIV-1 envelope epitopes targeted by antibody-dependent cell-mediated cytotoxicity. J Virol 405 88:2633-2644.
17. Zhao Q, Ma L, Jiang S, Lu H, Liu S, He Y, Strick N, Neamati N, Debnath A K. 2005. Identification of N-phenyl-N′-(2,2,6,6-tetramethyl-piperidin-4-yl)-oxalamides as a new class of HIV-1 entry inhibitors that prevent gp120 binding to CD4. Virology 339:213-225.
18. Courter J R, Madani N, Sodroski J, Schon A, Freire E, Kwong P D, Hendrickson W A, Chaiken I M, LaLonde J M, Smith A B, 3rd. 2014. Structure-based design, synthesis and validation of CD4-mimetic small molecule inhibitors of HIV-1 entry: conversion of a viral entry agonist to an antagonist. Acc Chem Res 47:1228-1237.
19. Melillo B, Liang S, Park J, Schon A, Courter J R, LaLonde J M, Wendler D J, Princiotto A M, Seaman M S, Freire E, Sodroski J, Madani N, Hendrickson W A, Smith A B, 3rd. 2016. Small-Molecule CD4-Mimics: Structure-Based Optimization of HIV-1 Entry Inhibition. ACS Med Chem Lett 7:330-334
20. Madani N, Princiotto A M, Schon A, LaLonde J, Feng Y, Freire E, Park J, Courter J R, Jones D M, Robinson J, Liao H X, Moody M A, Permar S, Haynes B, Smith A B, 3rd, Wyatt R, Sodroski J. 2014. CD4-mimetic small molecules sensitize human immunodeficiency virus to vaccine-elicited antibodies. J Virol 88:6542-6555.
21. Madani N, Princiotto A M, Schon A, LaLonde J, Feng Y, Freire E, Park J, Courter J R, Jones D M, Robinson J, Liao H X, Moody M A, Permar S, Haynes B, Smith A B, 3rd, Wyatt R, Sodroski J. 2014. CD4-mimetic small molecules sensitize human immunodeficiency virus to vaccine-elicited antibodies. J Virol 88:6542-6555.
22. Madani N, Princiotto A M, Zhao C, Jahanbakhshsefidi F, Mertens M, Herschhorn A, Melillo B, Smith A B, 3rd, Sodroski J. 2017. Activation and Inactivation of Primary Human Immunodeficiency Virus Envelope Glycoprotein Trimers by CD4-Mimetic Compounds. J Virol 91.
23. Ding S, Verly M M, Princiotto A, Melillo B, Moody T, Bradley T, Easterhoff D, Roger M, Hahn B H, Madani N, Smith Iii A B, Haynes B F, Sodroski J M D, Finzi A. 2016. Small Molecule CD4-Mimetics Sensitize HIV-1-infected Cells to ADCC by Antibodies Elicited by Multiple Envelope Glycoprotein Immunogens in Non-Human Primates. AIDS Res Hum Retroviruses doi:10.1089/AID.2016.0246.
24. Richard J, Prevost J, von Bredow B, Ding S, Brassard N, Medjahed H, Coutu M, Melillo B, Bibollet-Ruche F, Hahn B H, Kaufmann D E, Smith A B, 3rd, Sodroski J, Sauter D, Kirchhoff F, Gee K, Neil S J, Evans D T, Finzi A. 2017. BST-2 Expression Modulates Small CD4-Mimetic Sensitization of HIV-1-Infected Cells to Antibody-Dependent Cellular Cytotoxicity. J Virol 91.
25. Richard J, Veillette M, Brassard N, Iyer S S, Roger M, Martin L, Pazgier M, Schon A, Freire E, Routy J P, Smith A B, 3rd, Park J, Jones D M, Courter J R, Melillo B N, Kaufmann D E, Hahn B H, Permar S R, Haynes B F, Madani N, Sodroski J G, Finzi A. 2015. CD4 mimetics sensitize HIV-1-infected cells to ADCC. Proc Natl Acad Sci USA 112:E2687-2694.
26. Veillette M, Coutu M, Richard J, Batraville L A, Desormeaux A, Roger M, Finzi A. 2014. Conformational evaluation of HIV-1 trimeric envelope glycoproteins using a cell-based ELISA assay. J Vis Exp doi:10.3791/51995:51995.
27. Haim H, Strack B, Kassa A, Madani N, Wang L, Courter J R, Princiotto A, McGee K, Pacheco B, Seaman M S, Smith A B, 3rd, Sodroski J. 2011. Contribution of Intrinsic Reactivity of the HIV-1 Envelope Glycoproteins to CD4-Independent Infection and Global Inhibitor Sensitivity. PLoS Pathog 7:e1002101.
28. Haim H, Si Z, Madani N, Wang L, Courter J R, Princiotto A, Kassa A, DeGrace M, McGee-Estrada K, Mefford M, Gabuzda D, Smith A B, 3rd, Sodroski J. 2009. Soluble CD4 and CD4-mimetic compounds inhibit HIV-1 infection by induction of a short-lived activated state. PLoS Pathog 5:e1000360
29. Wyatt R, Moore J, Accola M, Desjardin E, Robinson J, Sodroski J. 1995. Involvement of the V1/V2 variable loop structure in the exposure of human immunodeficiency virus type 1 gp120 epitopes induced by receptor binding. J Virol 69:5723-5733.
30. Rizzuto C D, Wyatt R, Hernandez-Ramos N, Sun Y, Kwong P D, Hendrickson W A, Sodroski J. 1998. A conserved HIV gp120 glycoprotein structure involved in chemokine receptor binding. Science 280:1949-1953.
31. Zoubchenok D, Veillette M, Prevost J, Sanders-Buell E, Wagh K, Korber B, Chenine A L, Finzi A. 2017. Histidine 375 Modulates CD4 Binding in HIV-1 CRF01_AE Envelope Glycoproteins. J Virol 91.
32. Alsahafi N, Bakouche N, Kazemi M, Richard J, Ding S, Bhattacharyya S, Das D, Anand S P, Prevost J, Tolbert W D, Lu H, Medjahed H, Gendron-Lepage G, Ortega Delgado G G, Kirk S, Melillo B, Mothes W, Sodroski J, Smith A B, 3rd, Kaufmann D E, Wu X, Pazgier M, Rouiller I, Finzi A, Munro J B. 2019. An Asymmetric Opening of HIV-1 Envelope Mediates Antibody-Dependent Cellular Cytotoxicity. Cell Host Microbe 25:578-587 e575. 471
33. Richard J, Pacheco B, Gohain N, Veillette M, Ding S, Alsahafi N, Tolbert W D, Prevost J, Chapleau J P, Coutu M, Jia M, Brassard N, Park J, Courter J R, Melillo B, Martin L, Tremblay C, Hahn B H, Kaufmann D E, Wu X, Smith A B, 3rd, Sodroski J, Pazgier M, Finzi A. 2016. Co-receptor Binding Site Antibodies Enable CD4-Mimetics to Expose Conserved Anti-cluster A ADCC Epitopes on HIV-1 Envelope Glycoproteins. EBioMedicine doi:10.1016/j.ebiom.2016.09.004.
34. Veillette M, Coutu M, Richard J, Batraville L A, Dagher O, Bernard N, Tremblay C, Kaufmann D E, Roger M, Finzi A. 2015. The HIV-1 gp120 CD4-Bound Conformation Is Preferentially Targeted by Antibody-Dependent Cellular Cytotoxicity-Mediating Antibodies in Sera from HIV-1-Infected Individuals. J Virol 89:545-551
35. Decker J M, Bibollet-Ruche F, Wei X, Wang S, Levy D N, Wang W, Delaporte E, Peeters M, Derdeyn C A, Allen S, Hunter E, Saag M S, Hoxie J A, Hahn B H, Kwong P D, Robinson J E, Shaw G M. 2005. Antigenic conservation and immunogenicity of the HIV coreceptor binding site. J Exp Med 201:1407-1419.
36. Moore P L, Crooks E T, Porter L, Zhu P, Cayanan C S, Grise H, Corcoran P, Zwick M B, Franti M, Morris L, Roux K H, Burton D R, Binley J M. 2006. Nature of nonfunctional envelope proteins on the surface of human immunodeficiency virus type 1. J Virol 80:2515-2528.
37. Kassa A, Finzi A, Pancera M, Courter J R, Smith A B, 3rd, Sodroski J. 2009. Identification of a Human Immunodeficiency Virus (HIV-1) Envelope Glycoprotein Variant Resistant to Cold Inactivation. J Virol.
38. Desormeaux A, Coutu M, Medjahed H, Pacheco B, Herschhorn A, Gu C, Xiang S H, Mao Y, Sodroski J, Finzi A. 2013. The highly conserved layer-3 component of the HIV-1 gp120 inner domain is critical for CD4-required conformational transitions. J Virol 87:2549-2562.
39. Finzi A, Xiang S H, Pacheco B, Wang L, Haight J, Kassa A, Danek B, Pancera M, Kwong P D, Sodroski J. 2010. Topological layers in the HIV-1 gp120 inner domain regulate gp41 interaction and CD4-triggered conformational transitions. Mol Cell 37:656-667.
40. Pacheco B, Alsahafi N, Debbeche O, Prevost J, Ding S, Chapleau J P, Herschhorn A, Madani N, Princiotto A, Melillo B, Gu C, Zeng X, Mao Y, Smith A B, 3rd, Sodroski J, Finzi A. 2017. Residues in the gp41 Ectodomain Regulate HIV-1 Envelope Glycoprotein Conformational Transitions Induced by gp120-Directed Inhibitors. J Virol 91.
41. Ochsenbauer C, Edmonds T G, Ding H, Keele B F, Decker J, Salazar M G, Salazar-Gonzalez J F, Shattock R, Haynes B F, Shaw G M, Hahn B H, Kappes J C. 2012. Generation of Transmitted/Founder HIV-1 Infectious Molecular Clones and Characterization of Their Replication Capacity in CD4 T Lymphocytes and Monocyte-Derived Macrophages. J Virol 86:2715-2728.
42. Bar K J, Tsao C Y, Iyer S S, Decker J M, Yang Y, Bonsignori M, Chen X, Hwang K K, Montefiori D C, Liao H X, Hraber P, Fischer W, Li H, Wang S, Sterrett S, Keele B F, Ganusov V V, Perelson A S, Korber B T, Georgiev I, McLellan J S, Pavlicek J W, Gao F, Haynes B F, Hahn B H, Kwong P D, Shaw G M. 2012. Early low-titer neutralizing antibodies impede HIV-1 replication and select for virus escape. PLoS Pathog 8:e1002721.
43. Parrish N F, Gao F, Li H, Giorgi E E, Barbian H J, Parrish E H, Zajic L, Iyer S S, Decker J M, Kumar A, Hora B, Berg A, Cai F, Hopper J, Denny T N, Ding H, Ochsenbauer C, Kappes J C, Galimidi R P, West A P, Jr., Bjorkman P J, Wilen C B, Doms R W, O'Brien M, Bhardwaj N, Borrow P, Haynes B F, Muldoon M, Theiler J P, Korber B, Shaw G M, Hahn B H. 2013. Phenotypic properties of transmitted founder HIV-1. Proc Natl Acad Sci USA 110:6626-6633.
44. Fenton-May A E, Dibben O, Emmerich T, Ding H, Pfafferott K, Aasa-Chapman M M, Pellegrino P, Williams I, Cohen M S, Gao F, Shaw G M, Hahn B H, Ochsenbauer C, Kappes J C, Borrow P. 2013. Relative resistance of HIV-1 founder viruses to control by interferon-alpha. Retrovirology 10:146.
45. Ding S, Veillette M, Coutu M, Prevost J, Scharf L, Bjorkman P J, Ferrari G, Robinson J E, Sturzel C, Hahn B H, Sauter D, Kirchhoff F, Lewis G K, Pazgier M, Finzi A. 2016. A Highly Conserved Residue of the HIV-1 gp120 Inner Domain Is Important for Antibody-Dependent Cellular Cytotoxicity Responses Mediated by Anti-cluster A Antibodies. J Virol 90:2127-2134.
46. Richard J, Prevost J, Baxter A E, von Bredow B, Ding S, Medjahed H, Delgado G G, Brassard N, Sturzel C M, Kirchhoff F, Hahn B H, Parsons M S, Kaufmann D E, Evans D T, Finzi A. 2018. Uninfected Bystander Cells Impact the Measurement of HIV-Specific Antibody-Dependent Cellular Cytotoxicity Responses. MBio 9.
47. Richard J, Veillette M, Ding S, Zoubchenok D, Alsahafi N, Coutu M, Brassard N, Park J, Courter J R, Melillo B, Smith A B, 3rd, Shaw G M, Hahn B H, Sodroski J, Kaufmann D E, Finzi A. 2016. Small CD4 Mimetics Prevent HIV-1 Uninfected Bystander CD4+T Cell Killing Mediated by Antibody-dependent Cell-mediated Cytotoxicity. EBioMedicine 3:122-134.
48. Madani N, Princiotto A M, Easterhoff D, Bradley T, Luo K, Williams W B, Liao H X, Moody M A, Phad G E, Vazquez Bernat N, Melillo B, Santra S, Smith A B, 3rd, Karlsson Hedestam G B, Haynes B, Sodroski J. 2016. Antibodies Elicited by Multiple Envelope Glycoprotein Immunogens in Primates Neutralize Primary Human Immunodeficiency Viruses (HIV-1) Sensitized by CD4-Mimetic Compounds. J Virol 90:5031-5046.
49. Kwong P D, Doyle M L, Casper D J, Cicala C, Leavitt S A, Majeed S, Steenbeke T D, Venturi M, Chaiken I, Fung M, Katinger H, Parren P W, Robinson J, Van Ryk D, Wang L, Burton D R, Freire E, Wyatt R, Sodroski J, Hendrickson W A, Arthos J. 2002. HIV-1 evades antibody-mediated neutralization through conformational masking of receptor-binding sites. Nature 420:678-682.
50. Wyatt R, Kwong P D, Desjardins E, Sweet R W, Robinson J, Hendrickson W A, Sodroski J G. 1998. The antigenic structure of the HIV gp120 envelope glycoprotein. Nature 393:705-711.
51. Moody M A, Gao F, Gurley T C, Amos J D, Kumar A, Hora B, Marshall D J, Whitesides J F, Xia S M, Parks R, Lloyd K E, Hwang K K, Lu X, Bonsignori M, Finzi A, Vandergrift N A, Alam S M, Ferrari G, Shen X, Tomaras G D, Kamanga G, Cohen M S, Sam N E, Kapiga S, Gray E S, Tumba N L, Morris L, Zolla-Pazner S, Gorny M K, Mascola J R, Hahn B H, Shaw G M, Sodroski J G, Liao H X, Montefiori D C, Hraber P T, Korber B T, Haynes B F. 2015. Strain-Specific V3 and CD4 Binding Site Autologous HIV-1 Neutralizing Antibodies Select Neutralization-Resistant Viruses. Cell Host Microbe 18:354-362.
52. Fontaine J, Chagnon-Choquet J, Valcke H S, Poudrier J, Roger M. 2011. High expression levels of B lymphocyte stimulator (BLyS) by dendritic cells correlate with HIV-related B-cell disease progression in humans. Blood 117:145-155.
53. Fontaine J, Coutlee F, Tremblay C, Routy J P, Poudrier J, Roger M. 2009. HIV infection affects blood myeloid dendritic cells after successful therapy and despite nonprogressing clinical disease. J Infect Dis 199:1007-1018.
54. International HIVCS, Pereyra F, Jia X, McLaren P J, Telenti A, de Bakker P I, Walker B D, Ripke S, Brumme C J, Pulit S L, Carrington M, Kadie C M, Carlson J M, Heckerman D, Graham R R, Plenge R M, Deeks S G, Gianniny L, Crawford G, Sullivan J, Gonzalez E, Davies L, Camargo A, Moore J M, Beattie N, Gupta S, Crenshaw A, Burtt N P, Guiducci C, Gupta N, Gao X, Qi Y, Yuki Y, Piechocka-Trocha A, Cutrell E, Rosenberg R, Moss K L, Lemay P, O'Leary J, Schaefer T, Verma P, Toth I, Block B, Baker B, Rothchild A, Lian J, Proudfoot J, Alvino D M, Vine S, Addo M M, et al. 2010. The major genetic determinants of HIV-1 control affect HLA class I peptide presentation. Science 330:1551-1557.
55. Kamya P, Boulet S, Tsoukas C M, Routy J P, Thomas R, Cote P, Boulassel M R, Baril J G, Kovacs C, Migueles S A, Connors M, Suscovich T J, Brander C, Tremblay C L, Bernard N. 2011. Receptor-ligand requirements for increased NK cell polyfunctional potential in slow progressors infected with HIV-1 coexpressing KIR3DL1*h/*y and HLA-581 B*57. J Virol 85:5949-5960.
56. Peretz Y, Ndongala M L, Boulet S, Boulassel M R, Rouleau D, Cote P, Longpre D, Routy J P, Falutz J, Tremblay C, Tsoukas C M, Sekaly R P, Bernard N F. 2007. Functional T cell subsets contribute differentially to HIV peptide-specific responses within infected individuals: correlation of these functional T cell subsets with markers of disease progression. Clin Immunol 124:57-68.
57. Alsahafi N, Debbeche 0, Sodroski J, Finzi A. 2015. Effects of the 1559P gp41 change on the conformation and function of the human immunodeficiency virus (HIV-1) membrane envelope glycoprotein trimer. PLoS One 10:e0122111.
58. Robinson J E Y H, Holton D, Elliott S, Ho D D. 1992. Distinct antigenic sites on HIV gp120 identified by a panel of human monoclonal antibodies. Journal of Cellular Biochemistry Supplement 16E:Q449.
59. Moore J P, Willey R L, Lewis G K, Robinson J, Sodroski J. 1994. Immunological evidence for interactions between the first, second, and fifth conserved domains of the gp120 surface glycoprotein of human immunodeficiency virus type 1. J Virol 68:6836-6847.
60. Richard J, Veillette M, Batraville L A, Coutu M, Chapleau J P, Bonsignori M, Bernard N, Tremblay C, Roger M, Kaufmann D E, Finzi A. 2014. Flow cytometry-based assay to study HIV-1 gp120 specific antibody-dependent cellular cytotoxicity responses. J Virol Methods 208:107-114.
61. Richard J. P J, Baxter A E., von Bredow B., Ding S., Medjahed H., Delgado G G., Brassard N., Stürzel CM., Kirchhoff F., Hahn B H., Parsons M S., Kaufmann D E., Evans D T., Finzi A. 2018. Uninfected Bystander Cells Impact the Measurement of HIV-Specific ADCC Responses. mBio.
62. Otwinowski Z, Minor W. 1997. Processing of X-ray diffraction data collected in oscillation mode. Methods Enzymol 276:307-326.
63. Collaborative Computational Project N. 1994. The CCP4 suite: programs for protein crystallography. Acta Crystallogr D Biol Crystallogr 50:760-763.
64. Emsley P, Cowtan K. 2004. Coot: model-building tools for molecular graphics. Acta Crystallogr D Biol Crystallogr 60:2126-2132.
65. Adams P D, Grosse-Kunstleve R W, Hung L W, Ioerger T R, McCoy A J, Moriarty N W, Read R J, Sacchettini J C, Sauter N K, Terwilliger T C. 2002. PHENIX: building new software for automated crystallographic structure determination. Acta Crystallogr D Biol Crystallogr 58:1948-1954.
66. Davis I W, Murray L W, Richardson J S, Richardson D C. 2004. MOLPROBITY: structure validation and all-atom contact analysis for nucleic acids and their complexes. Nucleic Acids Res 32:W615-619.

What is claimed:

1. A composition comprising:
(i) a mutated HIV-1 gp120 polypeptide from an HIV-1 strain, wherein the native residues at positions 61, 105, 108, 375, 474, 475 and 476 in the HIV-1 strain are substituted, and wherein
(a) the HIV-1 strain is a CRF01_AE strain and the native residues at positions 61, 105, 108, 375, 474, 475 and 476 are H, Q, V, H, N, I and K, respectively;
(b) the HIV-1 strain is a Clade A, B, C, D, G or H strain, and the native residues at positions 61, 105, 108, 375, 474, 475 and 476 are Y, H, I, S, D, M and R, respectively;
(c) the HIV-1 strain is a Clade F strain, and the native residues at positions 61, 105, 108, 375, 474, 475 and 476 are Y, H, I, S, N, M and K, respectively;
(d) the HIV-1 strain is a Clade J strain, and the native residues at positions 61, 105, 108, 375, 474, 475 and 476 are Y, H, I, S, D, M and K, respectively; or
(e) the HIV-1 strain is a Clade K strain, and the native residues at positions 61, 105, 108, 375, 474, 475 and 476 are Y, H, I, I, D, M and R, respectively;
and
(ii) a gp120 or gp41 ligand.

2. The composition of claim 1, wherein the HIV-1 strain is a CRF01_AE strain and the mutated HIV-1 gp120 polypeptide comprises one or more of the following substitutions: H61Y, Q105H, V1081, H375T or H375S, N474D, 1475M, and K476R.

3. The composition of claim 2, wherein the mutated HIV-1 gp120 polypeptide comprises the following substitutions: (a) (1) H61Y, (2) Q105H, (3) V1081, (4) H375T, (5) N474D, (6) 1475M, and (7) K476R; or (b) (1) H61Y, (2) Q105H, (3) V1081, (4) H375S, (5) N474D, (6) 1475M, and (7) K476R.

4. (canceled)

5. The composition of claim 1, wherein the HIV-1 strain is a clade A, B, C, D, G or H HIV-1 strain, and the mutated HIV-1 gp120 polypeptide comprises the following substitutions: Y61H, H105Q, 1108V, S375H, D474N, M4751, and R476K.

6. The composition of claim 1, wherein the HIV-1 strain is a clade F HIV-1 strain, and the mutated HIV-1 gp120 polypeptide comprises the following substitutions: Y61H, H105Q, 1108V, S375H, N474D, M4751, and K476R.

7. The composition of claim 1, wherein the HIV-1 strain is a clade J HIV-1 strain, and the mutated HIV-1 gp120 polypeptide comprises the following substitutions: Y61H, H105Q, 1108V, S375H, D474N, M4751, and K476R.

8. The composition of claim 1, wherein the HIV-1 strain is a clade K HIV-1 strain, and the mutated HIV-1 gp120 polypeptide comprises the following substitutions: Y61H, H105Q, 1108V, 1375H, D474N, M4751, and R476K.

9. The composition of claim 1, wherein the mutated HIV-1 gp120 polypeptide is an HIV envelope trimer.

10. The composition of claim 1, wherein the gp120 ligand induces said Env trimer into an open state 2/3 conformation.

11. The composition of claim 10, wherein the gp120 ligand is a CD4 mimetic (CD4mc).

12. The composition of claim 11, wherein said CD4mc is the following compound:

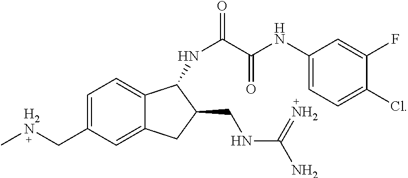

13. The composition of claim 1, wherein the gp120 ligand induces said Env trimer into a closed state 1 conformation.

14. The composition of claim 13, wherein the gp120 ligand is a conformational blocker.

15. The composition of claim 13, wherein the gp120 ligand is one of the following compounds:

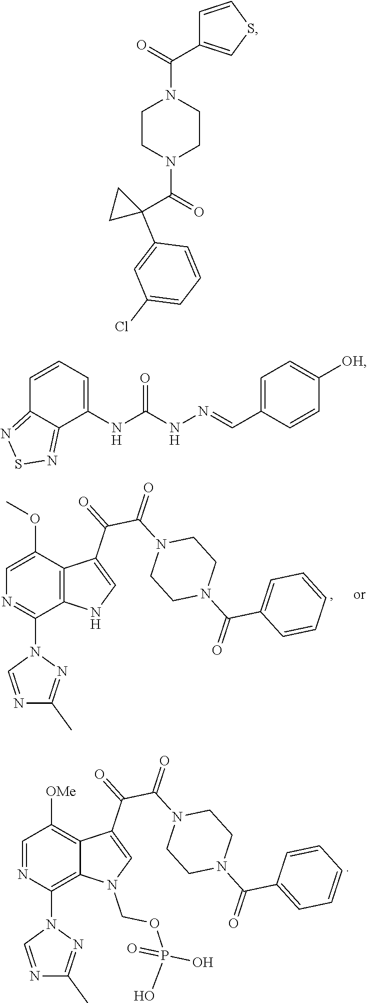

16. (canceled)

17. The composition of claim 1, wherein said mutated HIV-1 gp120 polypeptide is comprised in a cell, a liposome or a virus-like particle (VLP).

18. A method for eliciting an immune response to HIV-1 in a subject, comprising administering to the subject a prophylactically or therapeutically effective amount of (i) the mutated HIV-1 gp120 polypeptide defined in claim 1, and (ii) a gp120 ligand.

19-21. (canceled)

22. A method for determining whether a test agent binds to an HIV Env trimer into an open state 2/3 conformation comprising contacting said test agent with the mutated HIV-1 gp120 polypeptide defined in claim 1, and a gp120 ligand that induces said Env trimer into an open state 2/3 conformation.

23. A method for determining whether a test agent binds to an HIV Env trimer into a closed state 1 conformation comprising contacting said test agent with the mutated HIV-1 gp120 polypeptide defined in claim 1, and a gp120 ligand that induces said Env trimer into a closed state 1 conformation.

24. A method for inducing an HIV Env trimer into an open state 2/3 or closed state 1 conformation comprising contacting an HIV Env trimer comprising the mutated HIV-1 gp120 polypeptide defined in claim 1 with a gp120 ligand that induces said Env trimer into an open state 2/3 or closed state 1 conformation.

25-27. (canceled)

28. A method for determining whether a test agent induces a closed (state 1) or open (state 2/3) conformation of an HIV Env trimer comprising (a) contacting the mutated HIV-1 gp120 polypeptide defined in claim 1 with said test agent, and (b) determining whether the HIV Env trimer is in a closed (state 1) or open (state 2/3) conformation.

29-32. (canceled)


SEQUENCE LISTING (16 sequences)

SEQ ID NO: 1 - protein, 7 aa, Human immunodeficiency virus
HQVHNIK

SEQ ID NO: 2 - protein, 7 aa, Human immunodeficiency virus
YHISDMR

SEQ ID NO: 3 - protein, 7 aa, Human immunodeficiency virus
YHISNMK

SEQ ID NO: 4 - protein, 7 aa, Human immunodeficiency virus
YHISDMK

SEQ ID NO: 5 - protein, 7 aa, Human immunodeficiency virus
YHIIDMR

SEQ ID NO: 6 - protein, 856 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 7 - protein, 845 aa, Human immunodeficiency virus
MRVMGIQRNCQHLLRWGTMILGMIIICSAAENLWVTVYYGVPVWKDAETTLFCASDAKAYETEMHNVWATHACVPTDPNPQEIHLENVTEEFNMWKNNMVEQMHTDIISLWDQSLKPCVKLTPLCVTLNCSNVNVTNNTTNTHEEEIKNCSFNMTTELRDKKQKVYSLFYRLDVVQINENNSNSSYRLINCNTSAITQACPKVSFEPIPIHYCAPAGFAILKCKDKEFNGTGPCKNVSTVQCTHGIKPVVSTQLLLNGSLAEEEVIIRSENITNNAKTIIVQLTKPVKINCTRPNNNTRKSIRIGPGQAFYATGDIIGDIRQAHCNVSRSEWNKTLQKVAKQLRKYFKNKTIIFTNSSGGDLEITTHSFNCGGEFFYCNTSGLFNSTWNNGTMKNTITLPCRIKQIINMWQRAGQAMYAPPIQGVIRCESNITGLLLTRDGGNNNTNETFRPGGGDMRDNWRSELYKYKVVKIEPLGVAPTRAKRRVVEREKRAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQNEIWDNMTWLQWDKEISNYTHIIYNLIEESQNQQEKNEQDLLALDKWANLWNWFDISNWLWYIKIFIMIVGGLIGLRIVFAVLSVINRVRQGYSPLSFQTHTPNPRGLDRPGRIEEEGGEQGRDRSIRLVSGFLALAWDDLRSLCLFSYHRLRDFILIAARTVELLGHSSLKGLRLGWEGLKYLWNLLLYWGRELKISAINLVDTIAIAVAGWTDRVIEIGQRIGRAILHIPRRIRQGLERALL

SEQ ID NO: 8 - protein, 843 aa, Human immunodeficiency virus
MRVMGTQRNYQHLWRWGILILGMLIMCKATDLWVTVYYGVPVWKDADTTLFCASDAKAYDTEVHNVWATHACVPTDPNPQEVNLENVTEDFNMWKNNMVEQMHEDIISLWDQSLKPCVKLTPLCVTLNCSNANTTNNSTMEEIKNCSYNITTELRDKTQKVYSLFYKLDVVQLDESNKSEYYYRLINCNTSAITQACPKVSFEPIPIHYCAPAGFAILKCKDPRFNGTGSCNNVSSVQCTHGIKPVASTQLLLNGSLAEGKVMIRSENITNNAKNIIVQFNKPVPITCIRPNNNTRKSIRFGPGQAFYTNDIIGDIRQAHCNINKTKWNATLQKVAEQLREHFPNKTIIFTNSSGGDLEITTHSFNCGGEFFYCNTTGLFNSTWKNGTTNNTEQMITLPCRIKQIINMWQRVGRAMYAPPIAGVIKCTSNITGIILTRDGGNNETETFRPGGGDMRDNWRSELYKYKVVKIEPLGVAPTRAKRRVVEREKRAVGMGAVFLGFLGAAGSTMGAASITLTVQARQLLSGIVQQQSNLLKAIEAQQHLLKLTVWGIKQLQARVLALERYLQDQQLLGIWGCSGKLICATTVPWNSSWSNKTQEEIWNNMTWLQWDKEISNYTNIIYKLLEESQNQQEKNEQDLLALDKWANLWNWFNITNWLWYIRIFIMIVGGLIGLRIVIAIISVVNRVRQGYSPLSFQIPTPNPEGLDRPGRIEEGGGEQGRDRSIRLVSGFLALAWDDLRSLCLFSYHRLRDCILIAARTVELLGHSSLKGLRLGWEGLKYLWNLLLYWGRELKNSAISLLDTIAVAVAEWTDRVIEIGQRACRAILNIPRRIRQGFERALL

SEQ ID NO: 9 - protein, 841 aa, Human immunodeficiency virus
MRVKGIRKNYQHLWRWGTMLLGMLMICSAAEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLMNATNTNTTIIYRWRGEIKNCSFNITTSIRDKVQKEYALFYKLDVVPIDNDNTSYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSIHIGPGRAFYTTGEIIGDIRQAHCNISRAKWNNTLKQIVKKLREQFGNKTIVFNQSSGGDPEIVMHSFNCGGEFFYCNTTQLFNSTWNGTWNNTEGNITLPCRIKQIINMWQEVGKAMYAPPIRGQIRCSSNITGLLLTRDGGNNETEIFRPGGGDMRDNWRSELYKYKVVKIEPLGVAPTKAKRRVVQREKRAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTAVPWNASWSNKSLDEIWDNMTWMEWEREIDNYTSLIYTLIEESQNQQEKNEQELLELDKWASLWNWFDITNWLWYIKIFIMIVGGLVGLRIVFAVLSIVNRVRQGYSPLSFQTRLPAPRGPDRPEGIEEEGGERDRDRSGRLVDGFLALIWDDLRSLCLFSYHRLRDLLLIVTRIVELLGRRGWEVLKYWWNLLQYWSQELKNSAVSLLNATAIAVAEGTDRVIEVVQRACRAILHIPRRIRQGLERALL

SEQ ID NO: 10 - protein, 835 aa, Human immunodeficiency virus
MRVRGILRNCQQWWIWGILGFWMLMICNVVGNLWVTVYYGVPVWKEAKTTLFCASDAKAYEKEVHNVWATHACVPTDPNPQEIVLENVTENFNMWKNDMVDQMHEDIISLWDQSLKPCVKLTPLCVTLNCTNATNATNTMGEIKNCSFNITTELRDKKQKVYALFYRLDIVPLNENNSYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISEDKWNKTLQKVSKKLKEHFPNKTIKFEPSSGGDLEITTHSFNCRGEFFYCNTSKLFNSTYNSTNSTITLPCRIKQIINMWQEVGRAMYAPPIAGNITCKSNITGLLLTRDGGKNNTETFRPGGGDMRDNWRSELYKYKVVEIKPLGIAPTKAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILIAARAVELLGRSSLRGLQRGWEALKYLGSLVQYWGLELKKSAISLLDTIAIAVAEGTDRIIELIQRICRAIRNIPRRIRQGFEAALQ

SEQ ID NO: 11 - protein, 840 aa, Human immunodeficiency virus
MRVRGIQRNYQHLWRWGIMLLGMLMICSVAENLWVTVYYGVPVWKEATTTLFCASDAKSYKTEAHNIWATHACVPTDPNPQEIELENVTENFNMWKNNMVEQMHEDIISLWDQSLKPCVKLTPLCVTLNCTDVKRNNTSNDTNEGEMKNCSFNITTEIRDKKKQVHALFYKLDVVPIDDNNSNTSYRLINCNTSAITQACPKVTFEPIPIHYCAPAGFAILKCKDKKFNGTGPCKNVSTVQCTHGIRPVVSTQLLLNGSLAEEEIIIRSENLTNNAKIIIVQLNESVTINCTRPYNNTRQRTPIGPGQALYTTRIKGDIRQAHCNISRAEWNKTLQQVAKKLGDLLNKTTIIFKPSSGGDPEITTHSFNCGGEFFYCNTSRLFNSTWNNTKWNSTGKITLPCRIKQIINMWQGVGKAMYAPPIEGLIKCSSNITGLLLTRDGGANNSHNETFRPGGGDMRDNWRSELYKYKVVKIEPLGVAPTRAKRRVVEREKRAIGLGAMFLGFLGAAGSTMGAASMTLTVQARQLLSGIVQQQNNLLRAIEAQQHLLQLTVWGIKQLQARILAVERYLKDQQLLGIWGCSGKHICTTTVPWNSSWSNKSLDEIWNNMTWMEWEREIDNYTGLIYSLIEESQNQQEKNEQELLELDKWASLWNWFSITQWLWYIKIFIMIVGGLIGLRIVFAVLSLVNRVRQGYSPLSFQTLLPAPRGPDRPEGIEEEGGEQGRGRSIRLVNGFSALIWDDLRNLCLFSYHRLRDLILIAARIVELLGRRGWEALKYLWNLLQYWIQELKNSAISLFDTTAIAVAEGTDRVIEIVQRACRAILNIPTRIRQGLERALL

SEQ ID NO: 12 - protein, 832 aa, Human immunodeficiency virus
MRVRGMQRNWQHLGKWGLLFLGILIICNAAENLWVTVYYGVPVWKEATTTLFCASDAKSYEKEVHNVWATHACVPTDPNPQEVVLENVTENFDMWKNNMVEQMHTDIISLWDQSLKPCVKLTPLCVTLNCTDVNATNNDTNDNKTGAIQNCSFNMTTEVRDKKLKVHALFYKLDIVPISNNNSKYRLINCNTSTITQACPKVSWDPIPIHYCAPAGYAILKCNDKRFNGTGPCKNVSTVQCTHGIKPVVSTQLLLNGSLAEEDIIIRSQNISDNAKTIIVHLNESVQINCTRPNNNTRKSIHLGPGQAFYATGEIIGDIRKAHCNISGTQWNKTLEQVKAKLKSHFPNKTIKFNSSSGGDLEITMHSFNCRGEFFYCNTSGLFNDTGSNGTITLPCRIKQIVNMWQEVGRAMYAAPIAGNITCNSNITGLLLTRDGGQNNTETFRPGGGNMKDNWRSELYKYKVVEIEPLGVAPTKAKRQVVKRERRAVGIGAVFLGFLGAAGSTMGAASITLTVQARQLLSGIVQQQNNLLRAIEAQQHLLQLTVWGIKQLQARVLAVERYLKDQQLLGLWGCSGKLICTTNVPWNSSWSNKSQDEIWNNMTWMEWEKEISNYSNIIYRLIEESQNQQEKNEQELLALDKWASLWNWFDISNWLWYIKIFIMIVGGLIGLRIVFAVLSIVNRVRKGYSPLSLQTLIPSPREPDRPEGIEEGGGEQGKDRSVRLVNGFLALVWDDLRNLCLFSYRHLRDFILIAARIVDRGLRRGWEALKYLGNLTQYWSQELKNSAISLLNTTAIVVAEGTDRVIEALQRAGRAVLNIPRRIRQGLERALL

SEQ ID NO: 13 - protein, 831 aa, Human immunodeficiency virus
MRVREMQRNWQHLGKWGLLFLGILIICNAADNLWVTVYYGVPVWKEATTTLFCASDAKAYEREVHNVWATYACVPTDPSPQELVLGNVTENFNMWKNNMVDQMHEDIISLWDQSLKPCVKLTPLCVTLNCTDVNVTINTTNVTLGEIKNCSFNITTEIKDKKKKEYALFYRLDVVPINNSIVYRLISCNTSTVTQACPKVSFEPIPIHYCAPAGFAILKCNDKKFNGTGLCRNVSTVQCTHGIRPVVSTQLLLNGSLAEEDIIIRSENISDNTKTIIVQFNRSVEINCTRPNNNTRKSIRIGPGRAFYATGDIIGDIRKAYCNINRTLWNETLKKVAEEFKNHFNITVTFNPSSGGDLEITTHSFNCRGEFFYCNTSDLFNNTEVNNTKTITLPCRIRQFVNMWQRVGRAMYAPPIAGQIQCNSNITGLLLTRDGGKNGSETLRPGGGDMRDNWRSELYKYKVVKIEPLGVAPTKAKRQVVQREKRAVGIGAVLLGFLGAAGSTMGAASITLTVQARQLLSGIVQQQSNLLKAIEAQQHLLQLTVWGIKQLQARILAVERYLKDQQLLGIWGCSGKLICTTNVPWNSSWSNKSQDEIWDNMTWMQWEKEISNYTDTIYRLIEDAQNQQEKNEQDLLALDKWDNLWSWFTITNWLWYIKIFIMIVGGLIGLRIVFAVLSVVNRVRQGYSPLSLQTLIPNPRGPERPGGIEEEGGEQDRDRSIRLVSGFLALAWDDLRSLCLFSYRHLRDFILIAARTVDMGLKRGWEALKYLWNLPQYWGQELKNSAISLLDTTAIAVAEGTDRIIEVLQRAGRAVLHIPRRIRQGFERALL

SEQ ID NO: 14 - protein, 842 aa, Human immunodeficiency virus
MRVKGIQRNWQHLWKWGTLILGLVIICSASNNLWVTVYYGVPVWEDADTTLFCASDAKAYSTERHNVWATHACVPTDPNPQEITLENVTENFNMWKNNMVEQMHEDIISLWDESLKPCVKLTPLCVTLNCTDVNVTNNNTNNTKKEIKNCSFNITTEIRDKKKKEYALFYRLDVVPINDNGNSSIYRLINCNVSTIKQACPKVTFDPIPIHYCAPAGFAILKCRDKKFNGTGPCKNVSTVQCTHGIKPVVSTQLLLNGSLAEEEIIIRSENITDNTKVIIVQLNETIEINCTRPNNNTRKSIRIGPGQAFYATGDIIGDIRQAHCNVSRTKWNEMLQKVKAQLKKIFNKSITFNSSSGGDLEITTHSFNCRGEFFYCNTSGLFNNSLLNSTNSTITLPCKIKQIVRMWQRVGQAMYAPPIAGNITCRSNITGLLLTRDGGNNNTETFRPGGGDMRDNWRSELYKYKIVKIKPLGVAPTRARRRVVEREKRAVGLGAVLLGFLGAAGSTMGAASITLTVQVRQLLSGIVQQQSNLLRAIEAQQHLLQLTVWGIKQLQARVLAVERYLKDQQLLGIWGCSGKLICTTNVPWNTSWSNKSYNEIWDNMTWIEWEREISNYTQQIYSLIEESQNQQEKNEQDLLALDKWASLWNWFDITKWLWYIKIFIMIVGGLIGLRIVFAVLSIVNRVRQGYSPLSFQTLTHHQREPDRPERIEEGGGEQDKDRSIRLVSGFLALAWDDLRSLCLFSYHRLRDFILIAARTVELLGRSSLKGLRLGWEGLKYLWNLLLYWGQELKNSAINLLDTIAIAVANWTDRVIEVAQRACRAILNIPRRIRQGLERALL

SEQ ID NO: 15 - protein, 839 aa, Human immunodeficiency virus
TRVMETQRNYPSLWRWGTLILGMLLICSAAGNLWVTVYYGVPVWKEAKTTLFCASDAKAYETEKHNVWATHACVPTDPNPQEMVLENVTENFNMWENDMVEQMHTDIISLWDQSLKPCVKLTPLCVTLDCSNVNTTNATNSRFNMQEELTNCSFNVTTVIRDKQQKVHALFYRLDVVPIDDNNSYQYRLINCNTSVITQACPKVSFEPIPIHYCAPAGFAILKCNNKTFNGTGPCTNVSTVQCTHGIRPVVSTQLLLNGSLAEEQVIIRSKNISDNTKNIIVQLNKPVEITCTRPNNNTRKSIHLGPGQAFYATGDIIGDIRQAHCNISGKKWNKTLHQVVTQLGKYFDNRTIIFKPHSGGDMEVTTHSFNCRGEFFYCNTSGLFNSSWTNSTNDTKNIITLPCRIKQIVNMWQRVGQAMYAPPIKGNITCVSNITGLILTFDEGNNTVTFRPGGGDMRDNWRSELYKYKVVKIEPLGVAPTEARRRVVEREKRAVGMGAFFLGFLGAAGSTMGAASITLTVQARQLLSGIVQQQSNLLRAIQAQQHMLQLTVWGIKQLQARVLAVERYLKDQQLLGIWGCSGKLICTTNVPWNSSWSNKSLDEIWDNMTWMEWDKQINNYTEEIYRLLEVSQTQQEKNEQDLLALDKWASLWNWFSITNWLWYIKIFIMIVGGLIGLRIIFAVLSIVNRVRQGYSPLSFQTLIPNPRGPDRPEGIEEEGGEQDRDRSVRLVNGFLPLVWDDLRSLCLFSYRLLRDLLLIVVRTVELLGRRGREALKYLWNLLQYWGQELKNSAINLLNTTAIAVAEGTDRIIEIVQRAWRAILHIPRRIRQGFERTLL

SEQ ID NO: 16 - protein, 854 aa, Human immunodeficiency virus
MRVKETQMNWPNLWKWGTLILGLVIICSASDNLWVTVYYGVPVWRDADTTLFCASDAKAHETEVHNVWATHACVPTDPNPQEIHLENVTENFNMWKNNMVEQMQEDVISLWDQSLKPCVKLTPLCVTLNCTNANLTNVNNITNVSNIIGNITNEVRNCSFNMTTELRDKKQKVHALFYKLDIVQIEDNNSYRLINCNTSVIKQACPKISFDPIPIHYCTPAGYAILKCNDKNFNGTGPCKNVSSVQCTHGIKPVVSTQLLLNGSLAEEEIIIRSENLTNNAKTIIVHLNKSVEINCTRPSNNTRTSITIGPGQVFYRTGDIIGDIRKAYCEINGTKWNEVLKQVTEKLKEHFNNKTIIFQPPSGGDLEITMHHFNCRGEFFYCNTTKLFNNTCIGNETMEGCNGTIILPCKIKQIINMWQGAGQAMYAPPISGRINCVSNITGILLTRDGGANNTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKFLGLWGCSGKIICTTAVPWNSTWSNRSFEEIWNNMTWIEWEREISNYTNQIYEILTESQNQQDRNEKDLLELDKWASLWNWFDITNWLWYIKIFIMIVGGLIGLRIIFAVLSIVNRVRQGYSPLSFQTPTHHQREPDRPERIEEGGGEQGRDRSVRLVSGFLALAWDDLRSLCLFSYHRLRDFILIAARTVELLGHSSLKGLRRGWEGLKYLGNLLLYWGQELKISAISLLDATAIAVAGWTDRVIEVAQGAWRAILHIPRRIRQGLERALL